(19) United States
(12) Patent Application Publication
(10) Pub. No.: US 2003/0068803 A1
(43) Pub. Date: Apr. 10, 2003

(54) Purification of functional ribonucleoprotein complexes

(76) Inventors: Robin Reed, Belmont, MA (US); Zhaolan Zhou, Malden, MA (US)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 10/047,991

(22) Filed: Jan. 14, 2002

Related U.S. Application Data
(60) Provisional application No. 60/261,521, filed on Jan. 12, 2001.

(51) Int. Cl. C12N 9/22; C12N 9/64

(52) U.S. Cl. 435/199; 435/226

(57) ABSTRACT

The invention provides methods and reagents for isolating functional ribonucleoprotein complexes, such as functional eukaryotic spliceosomal complexes. The methods and reagents can be used, e.g., in diagnostic methods for determining the presence of abnormal ribonucleoprotein complexes.

### CROSS-REFERENCE TO RELATED APPLICATIONS

This application claims the benefit of U.S. Provisional Application No. 60/261,521, filed Jan. 12, 2001, the contents of which are specifically incorporated by reference herein.

### GOVERNMENT SUPPORT

This invention was made with government support under grant No. GM43375 by the National Institutes of Health. The government has certain rights in the invention.

### BACKGROUND OF THE INVENTION

The association of cellular RNAs and proteins into large biologically important ribonucleoprotein (RNP) complexes was first demonstrated with the isolation and characterization of ribosomes, the sites of cellular protein synthesis (see, e.g. Nomura (1973) Science 179: 864-73). Since then, many other types of cellular ribonucleoprotein complexes have been recognized. It now appears that many ribonucleoprotein complexes form only transiently in vivo and are present in only minute quantities that make biochemical isolation and characterization difficult. Indeed, many biologically important RNA-protein interactions have only recently been recognized. For example, specific RNA binding proteins play a role in sex specific splicing in Drosophila (see Lynch & Maniatis (1996) Genes Dev 10: 2089-101) and in regulation of splicing in the retroviral life cycle (see Fogel & McNally (2000) J Biol Chem 275:32371-8). Other examples of biologically important RNA/protein complexes include: a ribonucleoprotein complex containing molecular chaperones, such as heat shock protein 90, which plays a role in reverse transcriptase function (see Hu & Anselmo (2000) J Virol 74: 11447-55); a large ribonucleoprotein complex containing FMRP (the Fragile-X Mental Retardation Protein) the absence of which is associated with fragile-X human genetic syndrome (Beaulieu (2000) Biochem Biophys Res Commun 275: 608-10); and a ribonucleoprotein complex which is critical to translationally regulated differentiation events occurring during spermatogenesis (Braun (2000) Int J Androl 23 Suppl 2: 92-4). Still other ribonucleoprotein complexes are involved in: ribosomal RNA maturation (Lalev et al. (2000) J Mol Biol 302: 65-77); protein secretion via the signal recognition particle (Westermann & Weber (2000) Biochim Biophys Acta 1492: 483-7); and chromosomal telomere formation and maintenance (Niu et al. (2000) Mol Cell Biol 20: 6806-15). Still other conserved cellular ribonucleoprotein structures have been observed, but their function remains unclear (see e.g. Kong et al. (2000) RNA 6: 890-900; discussing the conserved 13-MDa vault complex). A particularly significant role of ribonucleoproteins is in facilitating particular types of gene “splicing” reactions necessary for the removal of non-coding intronic sequences present in virtually all RNA pol II-encoded mammalian genes.

Eukaryotic nuclear pre-mRNA introns and group II introns splice by essentially similar mechanisms. The intron is excised as a lariat structure, and the two flanking exons are joined. Moreover, the chemistry of the two processes is similar. In both, a 2 hydroxyl group within the intron serves as the nucleophile to promote cleavage at the 5′ splice site, and the 3′ hydroxyl group of the upstream exon is the nucleophile that cleaves the 3′ splice site by forming the exon-exon bond. However, in contrast to the conserved structural elements that reside within group I and II introns, the only conserved features of nuclear pre-mRNA introns are restricted to short regions at or near the splice junctions. In yeast, these motifs are (i) a conserved hexanucleotide at the 5′ splice, (ii) an invariant heptanucleotide, the UACUAAC Box, surrounding the branch point A, (iii) a generally conserved enrichment for pyrimidine residues adjacent to the invariant AG dinucleotide at the 3′ splice site. Further characteristics of nuclear pre-mRNA splicing in vitro that distinguish it from autocatalytic splicing are the dependence on added cell-free extracts, and the requirement for adenosine triphosphate (ATP). Another key difference is that nuclear pre-mRNA splicing generally requires multiple small nuclear ribonucleoproteins (snRNPs) and other accessory proteins, which can make-up a larger multi-subimit complex (splicesome) that facilitates splicing. A large number of different ribonucleoprotein complexes are associated with the processing and export of pre-mRNAs into mature, cytoplasmic mRNAs. A critical step in the formation of mature mRNAs is the removal of noncoding intronic sequences from pre-mRNAs by the action of a large ribonucleoprotein complex termed the spliceosome. Spliceosomes appear to assemble through multiple dynamic interactions among at least five spliceosomal small nuclear RNAs (snNRAs), approximately 50 spliceosomal proteins and the pre-mRNA template.

During spliceosome assembly, multiple dynamic interactions occur among the five spliceosomal snRNAs (U1, U2, U4, U5, and U6), the ˜50 spliceosomal proteins and the pre-mRNA. These interactions take place during assembly of the spliceosomal complexes which form in the temporal order E, A, B and C. The E complex assembles in the absence of ATP whereas assembly of the other complexes is ATP-dependent. According to the present model for spliceosome assembly, U1 snRNP first binds in the E complex, followed by U2 snRNP binding in the A complex and U4/5/6 snRNP binding in the B complex. Several rearrangements then occur which activate the spliceosome for the two catalytic steps of splicing in the C complex (Burge, C. B. et al. (1998) In The RNA World, 2d ed. 525-60; Staley, J. P. et al. (1998) Cell 92:315-26; Reed, R. (2000) Cur. Opin. Cell Biol. v.12, issue 3).

It has not been possible to isolate spliceosomal complexes that are both highly purified and complete, e.g., functional. In many of the methods used to isolate spliceosomal complexes, high salt or heparin treatment is required (e.g. Hong, W. et al. (1997) Nucleic Acids Res. 25:354-61; Staknis, D. et al (1994) Mol. Cell Biol. 14; Bennett, M. et al. (1992) Genes Dev. 6:1986-2000; Staley, J. P. et al. (1999) Mol. Cell 3:55-64; Grabowski, P. J. et al. (1986) Science 233:1294-99; Konarska, M. M. et al. (1986) Cell 46:845-55; Zillmann, M. et al. (1988) Mol. Cell Biol. 8:814-21; Jamison, S. F. et al. (1992) Proc. Natl. Acad. Sci. USA 89:5482-86; Konarska, M. M. et al. (1987) Cell 49:763-74). A number of problems with these protocols exist. First, the splicing complexes become irreversibly bound to the affinity matrix so that active splicing complexes cannot be released. Furthermore, the previous method required that the spliceosomes be purified in the presence of a high salt concentration, however such high salt conditions inevitably result in the loss of some of the components of the spliceosomal RNP complex.

Accordingly it would be desirable to have a method for purifying complete, e.g., functional, ribonucleoprotein complexes, for use, e.g., in diagnostic assays.

### SUMMARY OF THE INVENTION

The invention provides methods and reagents for isolating ribonucleoprotein complexes that are both functional and highly purified. The method and reagents are generally applicable to the affinity purification of any ribonucleoprotein complex, especially those ribonucleoprotein complexes which interact with a specific RNA sequence.

In a preferred embodiment, the invention provides methods for forming an isolated ribonucleoprotein complex comprising: providing an RNA affinity substrate comprising a ribonucleoprotein assembly sequence and an affinity tag; contacting the RNA affinity substrate with a protein mixture so as to permit the formation of a ribonucleoprotein complex on said ribonucleoprotein assembly sequence; subjecting said ribonucleoprotein complex to chromatographic separation; and subjecting said ribonucleoprotein complex to affinity selection, wherein the affinity tag binds to an affinity matrix, thereby forming an isolated ribonucleoprotein complex. The method preferably comprises eluting said ribonucleoprotein complex from said affinity matrix by disrupting the interaction of the affinity tag with the affinity matrix. The ribonucleoprotein complex can be selected from the group consisting of a spliceosomal complex, an hnRNP complex, an mRNA export complex, an mRNA localization complex, an RNA editing complex, and an intron complex. The ribonucleoprotein complex can be a spliceosomal complex selected from the group consisting of an E complex, an A complex, a B complex and a C complex. For example, the ribonucleoprotein complex can be an H complex. The ribonucleoprotein assembly sequence can be selected from the group consisting of a pre-mRNA sequence, a 5′ splice site, a 3′ splice site, and an intronless RNA.

In a preferred embodiment, the affinity tag binds to an affinity matrix through the intermediate of a fusion protein comprising a polypeptide binding specifically to the affinity tag and a polypeptide binding specifically to the affinity matrix. The affinity tag may comprise at least one MS2 or R17 coat protein recognition site and the polypeptide binding specifically to the affinity tag is an MS2 or R17 coat protein or portion thereof sufficient for binding to the MS2 or R17 coat protein recognition site, respectively. The polypeptide binding specifically to the affinity matrix may be selected from the group consisting of a maltose binding protein; a 6× His peptide; glutathione S transferase; or portion thereof sufficient to bind specifically to an affinity matrix. In one embodiment, the polypeptide binding specifically to the affinity matrix is a maltose binding protein or portion thereof sufficient to bind to amylose, the affinity matrix is an amylose matrix, and the ribonucleoprotein complex is eluted from the amylose matrix with maltose or a maltose analog. The method may comprise contacting the RNA affinity substrate with the fusion protein, such that the fusion protein binds specifically to the affinity tag, prior to contacting the RNA affinity substrate with the protein mixture.

The protein mixture may be a eukaryotic cell nuclear extract or a subfraction thereof. In a preferred embodiment of the invention, the chromatographic separation is a gel filtration. In another preferred embodiment, the affinity selection is performed in a low ionic strength buffer, e.g., a low ionic strength buffer comprises a final salt concentration of less than about 100 mM.

The invention provides methods for isolating a spliceosome comprising: providing an RNA affinity substrate comprising a pre-mRNA sequence and an MS2 coat protein recognition site; contacting the RNA affinity substrate with a fusion protein comprising (i) an MS2 coat protein or portion thereof sufficient to bind specifically to the MS2 coat protein recognition site and (ii) a polypeptide binding specifically to a ligand, such that the fusion protein binds to RNA affinity substrate; contacting the RNA affinity substrate with a eukaryotic cell nuclear extract so as to permit the formation of a spliceosome mRNA complex; subjecting the spliceosome mRNA complex to chromatographic separation; and subjecting the spliceosome mRNA complex to affinity selection on an affinity matrix comprising the ligand, thereby isolating a spliceosome. In a preferred embodiment, the RNA affinity substrate comprises at least two MS2 coat protein recognition sites. The polypeptide binding specifically to a ligand may be selected from the group consisting of a maltose binding protein; a 6× His peptide; glutathione S transferase; or portion thereof sufficient to bind specifically to the ligand. The polypeptide binding specifically to a ligand may be a maltose binding protein or portion thereof sufficient to bind to amylose; and the affinity selection may comprise binding of the spliceosome mRNA complex on an amylose matrix and elution of the ribonucleoprotein complex from the amylose matrix with maltose or a maltose analog.

The invention further provides isolated spliceosome preparations, e.g., isolated by the method described above. In a preferred embodiment, more than about 10% of the pre-mRNA sequences associated with an isolated spliceosome complex can be chased into a completely spliced mRNA in a splicing reaction. Certain preferred spliceosome preparations comprise a quantitative amount of 17S U2 U2 small nuclear ribonucleoprotein (snRNP) and/or SP3a polypeptide. The spliceosome preparation may be an E or A complex spliceosome preparation.

The invention also provides ribonucleic acids comprising a ribonucleoprotein complex binding site and at least one phage coat protein recognition site. The ribonucleoprotein complex binding site may be a spliceosome binding site. The phage coat protein binding site may be an MS2 or R17 coat protein recognition site. The spliceosome binding site may be an adenovirus major late pre-mRNA. The invention also provides nucleic acids encoding such ribonucleic acids. The nucleic acids may be operably linked to an RNA promoter capable of transcribing the nucleic acid.

The invention also provides diagnostic assays for determining whether a subject has abnormal ribonucleoprotein complexes, comprising obtaining a sample of cells from a subject; purifying ribonucleoprotein complexes from the cells of the subject; and determining the presence in the purified ribonucleoprotein complexes of one or more proteins. A difference in the amount of one or more proteins in the ribonucleoprotein complexes of the subject relative to its amount in a corresponding normal ribonucleoprotein complex indicates that the subject has abnormal ribonucleoprotein complexes. In one embodiment, the invention provides a diagnostic assay for determining whether a subject has abnormal spliceosome complexes, comprising: obtaining a sample of cells from a subject; purifying spliceosome complexes from the cells of the subject; and determining whether the pre-mRNA sequence was spliced during the purification. Splicing of the pre-mRNA sequence indicates that the spliceosome complexes of the subject are functional, whereas the absence of splicing or the pre-mRNA indicates that the spliceosome complexes of the subject are not functional, thereby indicating that the subject has abnormal spliceosome complexes. Also within the scope of the invention are diagnostic kits comprising, e.g., at least two elements selected from the group consisting of an RNA affinity substrate; a fusion protein comprising an affinity tag binding polypeptide and a ligand binding polypeptide; a chromatographic separation reagent; and an affinity purification reagent.

Therapeutic methods are also within the scope of the invention. In one embodiment, the invention provides a method for treating a subject having a disorder associated with abnormal ribonucleoprotein complexes, comprising obtaining a sample of cells from a subject; purifyng ribonucleoprotein complexes from the cells of the subject; determining the presence in the purified ribonucleoprotein complexes of one or more proteins; and normalizing the amount of ribonucleoproteins in the subject, to thereby treat the subject having a disorder associated with abnormal ribonucleoproteins complexes.

The invention also provides methods for in vitro splicing of nucleic acids. The method may comprise contacting a pre-mRNA to be spliced with purified spliceosomes or a fraction thereof. The purified spliceosomes may be used, e.g., in trans splicing reactions, thereby generating splice variants.

### BRIEF DESCRIPTION OF THE FIGURES

FIG. 1 shows the purity and snRNA /protein composition of active spliceosomal E complexes using the method.

FIG. 2 shows that U2 snRNP is stoichiometrically associated with the E and A complexes.

FIG. 3 shows that U2 snRNP associates with the E complex in the absence of the BPS.

FIG. 4 shows SF3a immunodepletion and reconstitution with recombinant SF3a.

FIG. 5 shows that SF3a is functionally associated with the purified E complex.

FIG. 6 shows that SF3a is required for E complex assembly.

FIG. 7 depicts a model for the early steps in spliceosome assembly.

FIG. 8 shows the polypeptide and nucleic acid sequence of the MS2 phage coat protein binding sequence (SEQ ID NO: 1 and 2, respectively).

FIG. 9 shows the polypeptide sequence of the maltose binding protein (SEQ ID NO: 4) and the nucleotide sequence of E. coli K12 (GenBank Accession No. AE000476), the complement of which encodes the maltose binding protein (SEQ ID NO: 3).

### DETAILED DESCRIPTION OF THE INVENTION

The invention is based at least in part on the discovery of a method for forming isolated ribonucleoprotein complexes that are functional, such as spliceosomes that are capable of splicing pre-mRNA.

1. Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

An “affinity tag” is a portion of an RNA affinity substrate that is capable of binding to a molecule and thereby permit affinity purification of a molecule to which the affinity tag is linked. An affinity tag can be any molecule. In a preferred embodiment, an affinity tag is an RNA molecule.

An “abnormal ribonucleoprotein complex” is a complex that differs in the presence and/or amount of one or more proteins relative to that of a normal ribonuclear complex. A normal ribonuclear complex is one that is observed in most individuals, excluding individuals that are known to have abnormal ribonucleoprotein complexes. An abnormal ribonucleoprotein complex is a complex that is not functional, or that does not function adequately. For example an abnormal spliceosome complex may be one that is not functional, i.e., is not capable of splicing pre-mRNA.

A “chimeric polypeptide” or “fusion polypeptide” is a fusion of a first amino acid sequence encoding a first polypeptide with a second amino acid sequence encoding a second polypeptide.

A “disease associated with an abnormal ribonucleoprotein complex” refers to a disease that is characterized by the presence of an abnormal amount of one or more ribonucleoproteins in the complex, relative to that observed in normal ribonucleoprotein complexes. An abnormal amount of a protein can be, e.g., an undetectable amount of the absence of the protein. The disease may or may not be caused by the presence of an abnormal amount of one or more proteins. Exemplary diseases include fragile-X human genetic syndrome.

The term “equivalent” is understood to include nucleotide sequences encoding functionally equivalent polypeptides. Equivalent nucleotide sequences will include sequences that differ by one or more nucleotide substitutions, additions or deletions; and will, therefore, include sequences that differ from nucleotide sequences described herein or in the art, for example, due to the degeneracy of the genetic code.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the protein sequences of the present invention.

“Hybridization stringencies” are defined as follows. Appropriate stringency conditions which promote DNA hybridization, for example, 6.6×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. High stringency hybridization includes, e.g., hybridization at, e.g., 2×SSC at about 65° C., followed washing in about 0.2×SSC at about 55-65° C. Low stringency hybridization includes, e.g., hybridization at, e.g., 6×SSC at room temperature and washes in 2×SSC at room temperature. Moderately stringent conditions are, for example, about 2.0×SSC and about 40° C.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature. “Specific interaction” or “specific binding” between two molecules refers to an interaction that occurs predominantly between the two molecules, relative to the interaction of each with another molecule.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Normal ribonucleoprotein complexes” refers to those complexes observed in individuals not having abnormal ribonucleoprotein complexes, e.g., in individuals having functional ribonucleoprotein complexes.

“Normalizing the amount of a ribonucleoprotein” in a subject refers to modifying its level such as to bring it closer to that observed in normal ribonucleoprotein complexes.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. In comparing nucleotide and amino acid sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489. Other alignment algorithms and/or programs may be used, including FASTA, BLAST, or ENTREZ. FASTA and BLAST are available as a part of the GCG sequence analysis package (university of Wisconsin, Madison, Wis.), and can be used with, e.g., default settings. ENTREZ is available through the National Center for Biotechnology Information, National Library of Medicine, National Institutes of Health, Bethesda, Md. The percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative analysis uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors.

The terms “protein”, “polypeptide” and “peptide” are used interchangeably herein when referring to a gene product.

A “protein mixture” refers to a mixture of proteins, such as a cell lysate; a cell extract; a nuclear extract or fractions thereof; a mixture of purified or recombinant proteins; or a combination thereof.

A “quantitative amount” refers to an amount that is proportional to that of other proteins. For example, a “quantitative amount of an SP3a polypeptide” in a spliceosomal complex is an amount in the same range as that found in nature.

“RNA affinity substrate” refers to a nucleic acid or analog thereof or a nucleic acid linked to another molecule, comprising a ribonucleoprotein assembly sequence, and an affinity tag. In a preferred embodiment, an RNA affinity substrate is an RNA molecule.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a nucleic acid is under the control of a promoter sequence (or other transcriptional regulatory sequence) that controls the expression of the nucleic acid in the system used.

“Treating a disease” refers to preventing, curing or improving at least one symptom of the disease.

The term “vector” refers to a nucleic acid molecule capable of transporting another nucleic acid to which it has been linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

2. Methods and Reagents

In a preferred embodiment, the method of the invention provides means of forming an isolated ribonucleoprotein complex. The method preferably utilizes an RNA affinity substrate, which comprises both a ribonucleoprotein assembly sequence and an affinity tag. In a preferred embodiment, the RNA affinity substrate is contacted with a protein mixture containing ribonucleoproteins of interest, such as a mammalian nuclear extract containing spliceosome factors, so as to permit the formation of the particular ribonucleoprotein complex on the ribonucleoprotein assembly sequence. The assembled ribonucleoprotein complex is then preferably passed through a chromatographic separation step, such as a gel filtration step; and an affinity selection step. Without wanting to be limited to a particular mechanism of action, the affinity selection step allows the affinity tag present on the RNA affinity substrate to be bound to the affinity matrix so as to form an isolated ribonucleoprotein complex. In a preferred embodiment, the RNA affinity substrate is contacted with a fusion protein comprising a polypeptide binding specifically to the affinity tag and a polypeptide that is capable of binding specifically to a ligand affinity matrix prior to contacting the RNA affinity substrate with the protein mixture. In preferred embodiments, the method further provides for eluting the ribonucleoprotein complex from the affinity matrix by disrupting the interaction of the affinity tag with the affinity matrix.

The method is generally applicable to the purification of any ribonucleoprotein complex such as spliceosomal complexes, hnRNP complexes, mRNA export complexes, mRNA localization complexes, RNA editing complexes, telomerase complexes, fragile X protein complexes, reverse transcriptase complexes or gene silencing complexes. In preferred embodiments, the complex is a spliceosomal complex such as an E complex, an A complex, a B complex or a C complex. Alternatively pre-splicing complexes, such as an hnRNP complex (H complex) may also be isolated.

In a preferred embodiment, the RNA affinity substrate comprises a ribonucleoprotein assembly sequence and an affinity tag. An affinity tag is a molecule designed to facilitate purification. The RNA affinity substrate can be a nucleic acid, such as an RNA molecule. The RNA affinity substrate can also be a chimeric molecule comprising, e.g., an RNA portion and a DNA portion. In some embodiments, the ribonucleoprotein assembly sequence is a nucleic acid and the affinity tag is another molecule, e.g., a protein or a chemical compound. The ribonucleoprotein assembly sequence can be linked directly or indirectly to the affinity tag. For example, the ribonucleoprotein assembly sequence can be linked to the affinity tag through a linker molecule, e.g., an unrelated RNA sequence. The ribonucleoprotein assembly sequence can also be linked to the affinity tag through a chemical bond. The affinity tag sequence can be located 5′ or 3′ relative to the ribonucleoprotein assembly sequence, however, in preferred embodiments, the affinity tag is located 3′ of the ribonucleoprotein assembly sequence.

The ribonucleoprotein assembly sequence can be any sequence found in RNA to which specific proteins bind. The particular sequence used will depend on the type of ribonucleoprotein complex that one desires to isolate. Sequences to which such complexes, e.g., spliceosomal complexes, hnRNP complexes, mRNA export complexes, mRNA localization complexes, RNA editing complexes, telomerase complexes, fragile X protein complexes, reverse transcriptase complexes or gene silencing complexes, are known in the art. Spliceosomal RNA assembly sequences may be a pre-mRNA sequence or a portion of a pre-mRNA sequence such as an isolated exon-intron-exon sequence or a 5′ splice site (exon-intron junction) or a 3′ splice site (intron-exon junction). Sequences required for binding of certain types of splicesomes are described, e.g., in Michaud and Reed (1993) Genes & Dev. 7: 1008. Examples of pre-mRNA ribonucleoprotein assembly sequences and vectors encoding them include the adenovirus major late (pAdML) and Fushi Tarazu pre-mRNAs (Bennet et al. (1992) Genes & Dev. 6: 1986 and Luo et al. (1999) PNAS 96:14937); tropomyosin pre-mRNA (Bennet et al. (1992) Mol. Cell. Biol. 12:3165); β-globin (Bennet et al. (1992), supra); pAdMLΔ 3'ss (Michaud and Reed (1993) Genes Dev 7:1008-20); pAdMLΔAG and pAdMLPar (Gozani et al. (1994) EMBO J 13: 3356-67). Still other preferred sequences are described in the examples below. In general, preferred spliceosomal sequences contain all or a portion of a naturally occurring or synthetic intron sequence as described below. Alternatively an intronless RNA may be used for assembly.

The affinity tag can be any molecule that can be bound, directly or indirectly to a ligand, which binding is used during the affinity purification step of the ribonucleoprotein complex. In a preferred embodiment, the affinity tag is a nucleic acid, e.g., RNA, that comprises a sequence to which a protein or protein derivative binds, which protein or derivative either also binds to a ligand or interacts with, or is linked to, another molecule which binds to a ligand. For example, the affinity tag can be a sequence recognized by a fusion protein comprising a polypeptide binding specifically to the affinity tag (i.e., an “affinity tag binding polypeptide”) and a polypeptide binding specifically to the ligand (i.e., a “ligand binding polypeptide”). The affinity tag binding polypeptide and the ligand binding polypeptide can be fused directly to each other or alternatively through an intermediary peptide or chemical bond.

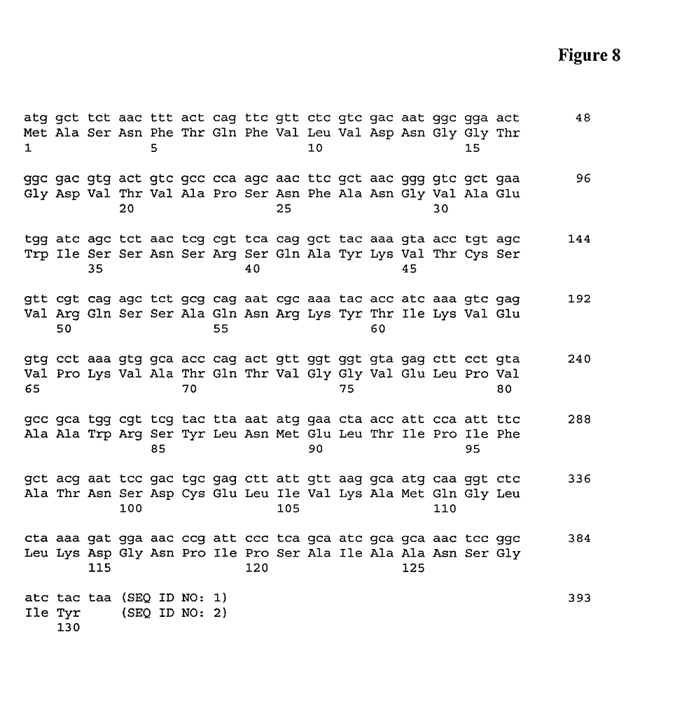

In a preferred embodiment, the affinity tag binding polypeptide is a polypeptide that binds specifically to an RNA sequence. In an even more preferred embodiment, the affinity tag polypeptide is a phage coat protein that binds single stranded RNA, such as the MS2 phage coat protein (see GenBank Accession No. J02467 M24961 V00642; De Wachter et al. (1971) Eur. J. Biochem. 22:400; Contreras et al. (1972) FEBS Letters 24:339; Jou et al. (1972) Nature 237:82; Jou et al. (1975) Nature 256:273; Van den Berghe et al. (1975) PNAS 72:2559; Fiers et al. (1976) Nature 260:500; Berzin et al. (1978) J. Mol. Biol. 119:101; Beremand et al. (1979) Cell 18:257; and Kastelein et al. (1982) Nature 295:35). The nucleotide sequence encoding MS2 phage coat protein is set forth in SEQ ID NO: 1 and FIG. 8.

The gene for MS2 coat protein can be obtained, e.g., by PCR amplification from pLexA-MS2 (SenGupta (1996) PNAS 93:8496) or from RNA obtained from MS2 phage, using the primers 5′-CAGGTCATATGGGTCCGCGGGCTTCTAACTTTACTCA GTTCGTT-3′ (SEQ ID NO: 5) and 5′-TGCTACTCGAGGGCGCTAGCGTAG ATGCCGGAGTTT GCTGCGAT-3′ (SEQ ID NO:6) and PFU polymerase (Stratagene).

The MS2 binding sequence (or recognition sequence) forms a specific hairpin structure and has the following sequence: 5′ CGTACACCATCAGGGTACG 3′ (SEQ IDNO: 7).

In another rembodiment, the affinity tag binding polypeptide is the Escherichia coli bacteriophage R17 coat protein, which binds to a short 21 nucleotide hairpin present in the R17 RNA genomic sequence that comprises the same binding sequence as that of MS2 binding sequence, i.e., SEQ ID NO: 7). Vectors encoding the RNA phage coat protein hairpin, and optimal conditions for binding to this sequence, have been described (see, e.g., Carey et al. (1983) Biochem 22: 2610-15; Bardwell and Wickens (1990) Nucl Acids Res 18: 6587-94; and Witherell et al. (1990) Biochem 29: 11051-57).

Other sequence-specific RNA binding proteins may also be used in the method of the invention. In particular, other sequence specific RNA binding proteins, useful for affinity-purification of RNAs, have been described (see e.g. Bardwell and Wickens (1990) Nucl Acids Res 18: 6587-94). Methods for the isolation of still other sequence specific RNA binding protein-binding sites have also been developed (see e.g. Bachler et al (1999) RNA 5: 1509-16).

A person of skill in the art will recognize that polypeptides which are analogs of the above-described affinity tag binding polypeptides can also be used, provided they bind sufficiently specifically to the affinity tag that they can be used in affinity purification. For example, polypeptides that differ from the above-recited polypeptides or any other RNA binding proteins in one or more amino acids can be used according to the invention. Such analogs may have one or more amino acid deletion, substitution, or addition. In certain embodiments, portions of RNA binding proteins can be used in the method of the invention, i.e., portions that are sufficient for providing specific binding to the affinity tag. Such portions can be identified according to methods known in the art, such as by conducting binding assays with various deletion mutants of the protein.

The affinity tag can comprise one or more affinity tag binding protein recognition sites. In certain embodiments, the affinity tag comprises at least 2, at least 3, at least 4, at least 5, 6, 7, 8, or 9 recognition sequences. In other embodiments, as many as 10 or more recognition sequences can be included in the affinity tag. In an illustrative embodiment, an affinity tag comprises at least one, preferably at least two and preferably at least three MS2 or R17 coat binding protein recognition sequences (i.e., hairpin structures).

Variants of the wild-type sequences, to which RNA binding proteins bind can also be used according to the invention. It has been shown, e.g., that sequences varying considerably from the R17 coat protein binding site can still bind the R17 coat protein (Romaniuk et al. (1987) Biochemistry 26:1563). A person of skill in the art can readily determine which variant sequences can still be bound by a particular RNA binding protein.

The ligand binding polypeptide can be any polypeptide binding sufficiently specifically to a ligand to allow affinity purification. In a preferred embodiment, the ligand binding polypeptide is maltose or a portion thereof sufficient to bind to a ligand. In an even more preferred embodiment, the ligand is amylose or an analog thereof, e.g., an analog that can bind to maltose binding protein. Maltose binding protein binds to amylose, and the interaction can be disrupted with maltose or a maltose analog. The amino acid sequence of maltose binding protein is the following:

(SEQ ID NO:4)

MKIKTGARILALSALTTMMFSASALAKIEEGKLVIWINGDKGYNGLAEVG
KKFEKDTGIKVTVEHPDKLEEKFPQVAATGDGPDIIFWAHDRFGGYAQSG
LLAEITPDKAGQDKLYPFTWDAVRYNKGLIAYPIAVEALSLIYNKDLLPN
PPKTWEEIPALDKELKAKGKSALMFNLQEPYFTWPLIAADGGYAFKYENG
KYDIKDVGVDNAGAKAGLTFLVDLIKNIKHMNADTDYSIAEAAFNKGETA
MTINGPWAWSMDTSKVNYGVTVLPTFKGQPSKPFVGVLSAGINAASPNKE
LAKEFLENYLLTDEGLEAVNKDKPLGAVALKSYEEELAKDPRIAATMENA
QKGEIMPNIPQMSAFWYAVRTAVINAASGRQTVDEALKDAQTRITK

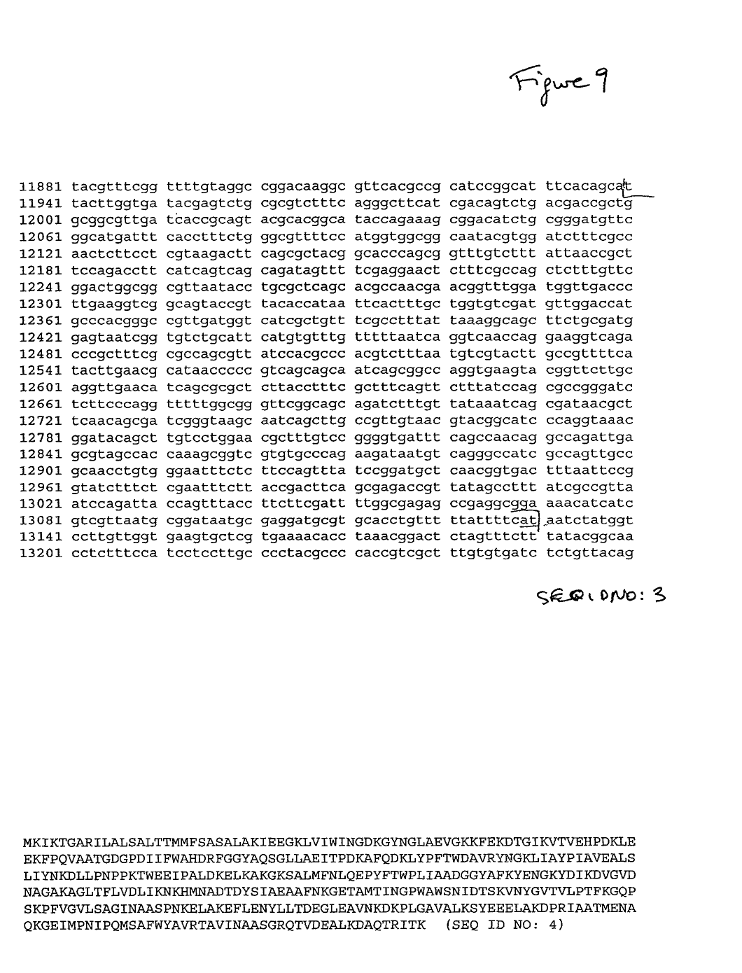

The mature protein consists of amino acids 27-396. The nucleic acid sequence for the maltose binding protein can be found, e.g., as GenBank Accession No. AE000476, SEQ ID NO: 3 and in FIG. 9. Maltose binding protein affinity reagents are available from New England Biolabs (see, e.g., www.neb.com/).

Other ligand binding polypeptides include those that can be used in immobilized metal affinity chromatography (IMAC). For example, a ligand binding polypeptide can be a polyhistidine sequence, for example, a hexahistidine sequence (6×His), which interacts specifically with metal ions such as zinc, nickel, or cobalt ions. It can also be a polylysine or polyarginine sequence, comprising at least about four lysine or four arginine residues, respectively, which interact specifically with zinc, copper or, for example a zinc finger protein. The sequences and affinity purification conditions are well known in the art. Vectors for producing fusion proteins contain such sequences and matrices to which they bind are commercially available. For example, the following kits provide vectors and matrices for purifying proteins containing His tags: QIAexpress Ni-NTA Protein Purification System of Qiagen (Qiagen, Calif.); HAT™ Protein Expression & Purification System (Clontech, Palo Alta, Calif.); pTrcHis Xpress™ Kit (InVitrogen); and BugBuster™ His.Bind® Purification Kit (Novagen).

In another embodiment, the ligand binding polypeptide is glutathione S transferase (GST) polypeptide, which can be prepared, e.g., by using pGEX prokaryotic expression vectors from Pharmacia (Piscataway, N.J.) When using GST fusion proteins, resin linked to GST (Sigma Chem. Co.; St. Louis, Mo., to glutathione or to an antibody specific for GST can be used, e.g., GST sepharose 4B colunm (Pharmacia-LKB) or mouse anti-GST-Sepharose® 4B, available from, e.g., Zymed Laboratories. Protein purification can be done as described, e.g., in Kuge et al. (1997) Protein Science 6: 1783 and in Tian et al. (1993) Cell 74:105. Systems for expressing and purifying recombinant proteins comprising a GST tag are available from Novagen as BugBuster™ GST.Bind™ Purification Kit and GST-Tag™ Assay Kit.

Yet other ligand binding polypeptides include a Self-Cleavable Chitin-binding Tag, e.g., as available from New England Biolabs as the IMPACT™-TWIN System and IMPACT™-CN System; a T7 tag are available from Novagen as T7.Tag™ Purification Kit; an S tag or thioredoxin (trxA), which are available from Novagen. Yet another ligand binding protein is a cellulose-binding protein A from Clostridium cellulovorans (see, eg., Shpigel et al. (2000) Biotechnol. Appl. Biochem. 31:197).

In other embodiments, the ligand binding protein and ligand pair consists of an antibody and an antigen to which the antibody binds. For example, the fusion protein binding to the affinity tag comprises an antigen and the affinity purification comprises using an antibody binding specifically to the antigen. In other embodiment, the fusion protein comprises an antibody (e.g., a single chain antibody) and the affinity purification comprises using an antigen to which the antibody binds specifically. In yet other methods, avidin and biotin are used.

In a preferred embodiment, a fusion protein comprises the MS2 coat protein and Maltose Binding Protein (MBP). In a preferred embodiment, the MS2 coat protein and/or MBP are full length. In an even more preferred embodiment, the MS2 coat protein and the MBP are full length. They are preferably fused directly to each other or with only a few amino acids between them. The MS2 is preferably fused to the C-terminus of MBP. In a preferred embodiment, the fusion protein consists of: full-length MBP-LVPRGSH-MRGSHHHHHH-full-length MS2 coat protein (SEQ ID NO: 8). The sequence “LVPRGSH” (SEQ ID NO: 9) is a thrombin cleavage site and “MRGSHHHHHH” (SEQ ID NO: 10) is a 6×His tag.

A person of skill in the art will recognize that polypeptides which are analogs of the above-described ligand binding polypeptides can also be used, provided they bind sufficiently specifically to the ligand that they can be used in affinity purification. For example, polypeptides that differ from the above-recited polypeptides or any other ligand binding proteins in one or more amino acids can be used according to the invention. Such analogs may have one or more amino acid deletion, substitution, or addition. For example, mutations within the maltose-binding cleft (W62E, A63E, Y155E, W230E, and W340E) have little or no effect on the solubility of fusion proteins comprising maltose binding protein. In contrast, three mutations near one end of the cleft (W232E, Y242E, and I317E) dramatically reduce the solubility of the same fusion proteins (Fox et al. (2001) Protein Sci 10:622). In certain embodiments, portions of ligand binding proteins can be used in the method of the invention, i.e., portions that are sufficient for providing specific binding to the ligand. Such portions can be identified according to methods known in the art, such as by conducting binding assays with various deletion mutants of the protein, e.g., as described in Fox et al., supra.

Accordingly, polypeptides used according to the invention, e.g., ligand binding polypeptides (e.g., maltose binding protein) and affinity tag binding polypetides (e.g., MS2 binding protein), can have an amino acid sequence or a nucleotide sequence encoding them that is at least about 70% identical, at least about 80%, 90%, 95%, 98% or 99% identical or homologous to amino acid or nucleotide sequences described herein or known in the art. Such polypeptides may have from 1 to about 5 amino acid substitutions; from about 5 to about 10; from about 10 to about 20 or from about 20 to about 50 amino acid substitutions, whether conservative amino acid substitutions or not. Polypeptides which are encoded by nucleic acids which hybridize, e.g., under stringent hybridization conditions, (e.g., with a wash in 0.2×SSC at 65° C.) to nucleic acids described herein or known in the art can also be used.

Affinity tag binding polypeptides and ligand binding polypeptides can be produced according to methods well known in the art, such as with prokaryotic or eukaroytic expression systems, as described, e.g., in the Examples. Following expression, the fusion proteins can be purified by affinity chromatography using the particular ligand to which they bind.

The RNA affinity substrates can be prepared according to methods known in the art. For, example, when the RNA affinity substrate is an RNA molecule, it can be synthesized in an in vitro transcription reaction, using, e.g., T7, T3, or SP6 RNA polymerases, as described, e.g., in Melton et al. (1984) Nucl. Acids Res. 12:7035. Reactions are also described in Gozani et al. (1994) EMBO J. 13:3356. Accordingly, in one embodiment, the RNA affinity substrate is synthesized by in vitro transcription of a DNA molecule encoding the RNA affinity substrate operably linked to a promoter, e.g., a viral RNA polymerase promoter, such as T7, T3 or SP6 promoter. The nucleic acid can be part of a vector or plasmid. Vectors that can be used for in vitro transcription of nucleic acid sequences can be obtained commercially from several companies. In one embodiment, a nucleic acid comprising an RNA affinity substrate sequence is inserted into a vector downstream of an RNA polymerase promoter. Prior to synthesis of RNA, the vector is linearized 3′ of the end of the RNA affinity substrate sequence. In a preferred embodiment, the invention provides plasmids encoding pre-mRNA or intronless mRNAs that contain 3 phage MS2 coat protein binding sites (hairpins) at the 3′ end of the RNA. Different restriction sites are included between the MS2 coat protein binding sites, such that cutting the plasmid with a restriction enzyme cutting the DNA at one of these sites generates DNA templates containing 1, 2, or 3 hairpins. Such an exemplary construct has the following nucleotide sequence:

(SEQ ID NO:9)

TAATACGACTCACTATAGGGAGACCGGCAGATCAGCTTGGCCGCGTCCAT
CTGGTCATCTAGGATCTGATATCATCGATGAATTCGAGCTCGGTACCCCG
TTCGTCCTCACTCTCTTCCGCATCGCTGTCTGCGAGGGCCAGCTGTTGGG
GTGAGTACTCCCTCTCAAAAGCGGGCATGACTTCTGCCCTCGAGTTATTA
ACCCTCACTAAAGGCAGTAGTCAAGGGTTTCCTTGAAGCTTTCGTGCTGA
CCCTGTCCCTTTTTTTTCCACAGCTGCAGGTCGACGTTGAGGACAAACTC
TTCGCGGTCTTTCCAGTACTCTTGGATCCGATATCCGTACACCATCAGGG
TACGAGCTAGCCCATGGCGTACACCATCAGGGTACGACTAGTAGATCTCG
TACACCATCAGGGTACGGAATTCTCTAGAGTCGAGTTCTATAGTGTCACC
TAAAT.

Fushi Tarazu (Ftz) pre-mRNA can also be used and is described, e.g. in Zhou et al. (2000) Nature 407:401.

RNA affinity substrates may be labeled prior to use, thereby permitting to follow the RNA and/or RNP complex, e.g., during purification. In a preferred embodiment, an RNA affinity substrate is labeled during its synthesis. For example, when the RNA affinity matrix is an RNA, it can be labeled during the in vitro transcription reaction. In one embodiment, transcription reactions are conducted in the presence of 10 μCi [³²P]UTP (800 Ci/mmol), 200 μm cold ATP, GTP, CTP and UTP, as described in Gozani et al. (1994) EMBO J. 13:3356.

The RNAs can be capped during transcription, as described, e.g., in Knonarska et al. (1984) Cell 38:731. The various methods employed in the preparation of the plasmids and transformation of host organisms are well known in the art. For other suitable expression systems, as well as general recombinant procedures, see Molecular Cloning A Laboratory Manual, 2^{nd} Ed., ed. by Sambrook, Fritsch and Maniatis (Cold Spring Harbor Laboratory Press: 1989).

In a preferred embodiment of the invention, an RNA affinity substrate is contacted with a fusion protein, comprising an affinity tag binding polypeptide and a ligand binding polypeptide, prior to contacting the RNA affinity substrate with a protein mixture. In an illustrative embodiment, the RNA affinity substrate and the fusion protein are incubated in a buffer containing 20 mM Hepes pH 7.0, 60 mM NaCl on ice for about 20 minutes, to allow the fusion protein to bind to the affinity tag of the RNA affinity substrate. Binding can be confirmed, e.g., by assaying an aliquot of the binding reaction on a native agarose gel, e.g., a 1.5% agarose gel.

In a preferred embodiment of the invention, an RNA affinity substrate is contacted with a protein mixture so as to permit the formation of a ribonucleoprotein complex on said ribonucleoprotein assembly sequence. The protein mixture used with the method of the invention may be a cell lysate or portion thereof. In a preferred embodiment, the protein mixture is a total eukaryotic cell nuclear extract or one or more subfractions thereof. The protein mixture can be composed of subfractions of eukaryotic nuclear extracts that have been fractionated chromatographically or immunodepleted of specific components using an antibody or antibodies. Protein mixtures and their preparation are described, e.g., in Krainer et al. (1984) Cell 36:993. In preferred embodiments, polyvinylalcohol (PVA) is omitted.

The cells can be obtained from a subject or they can be tissue culture cells. Where cells are from a subject, the cells can be any type of cells presumably having the desired ribonucleoprotein complex. For example, spliceosome complexes can be isolated from any nucleated cell, e.g., peripheral blood mononuclear cells (PBMCs). These can be isolated from a blood sample from a subject, and isolated as known in the art. Other cell samples can be obtained according to methods known in the art. The cells can be mammalian cells, e.g., cells from humans, non-human primates, ovines, bovines, porcines, equines, canines, and felines.

In a preferred embodiment, a nuclear extracts is prepared as follows. The cells are gently resuspended in hypotonic buffer, e.g., 10 mM HEPES, pH 7.9; 1.5 mM MgCl2; 10 mM KCl; 0.2 mM PMSF; 0.5 mM DTT, and then pelleted. The supernatant is poured off, and the cells are resuspended in hypotonic buffer. The cells are let swell for 10 minutes and then and steadily until 90% of the cells were lysed, as indicated, e.g., by trypan blue staining. The dounced cells are centrifuged and resuspended in low salt buffer, e.g., 20 mM HEPES, pH 7.9; 1.5 mM MgCl2; 20 mM KCl; 0.2 mM EDTA; 25% glycerol (v/v); 0.2 mM PMSF; 0.5 mM DTT. Approximately the same amount of high salt buffer is added as that of low salt buffer. High salt buffer may be, e.g., 20 mM HEPES, pH 7.9; 0.5 mM MgCl2; 1.5 M KCl; 0.2 mM EDTA; 25% glycerol (v/v); 0.2 mM PMSF; 0.5 mM DTT. The cells are rotated, e.g., e.g., for four hours at 0-4° C. The mixture is then centrifuged, e.g., at about 10K for about 30 the supernatant, which constitutes the nuclear extract, is pipetted into dialysis tubing and dialyzed for about 2 hours in buffer, e.g., 20 mM HEPES, pH 7.9; 100 mM KCl; 0.2 mM EDTA; 25% glycerol (v/v); 0.2 mM PMSF; 0.5 mM DTT. The buffer may be changed and dialysis continued for, e.g., another 2 hours. The nuclear extract is centrifuged at, e.g., 10K, and the supernatant removed. The nuclear extract is ready for use in the method of the invention. The nuclear extract can snap frozen in liquid nitrogen, and stored at −800.

In other embodiments, the protein mixture can be combined from several different cell extracts or fractions thereof. In yet other embodiments, one or more recombinantly expressed proteins are added to the protein mixture. A cell extract or nuclear extract can be prepared from any cell, either a cell line or a cell obtained from an animal. For example, an extract can be obtained from human cells, e.g., HeLa cells.

Large or small scale binding reactions can be conducted. For example, large scale reactions can be conducted in about 11 ml, containing, e.g., from about 20 to 50 μg RNA and about 30% nuclear extract (see, e.g., Bennett et al. (1992) Genes & Dev. 6:1986). Smaller reaction volumes of about 100 μl and may contain about 0.1 to 5 ng/μl of RNA, preferably about 0.2 to 4 ng/μl of RNA. The extract and the RNA affinity substrate can be incubated, e.g., at about 30° C. for about 30 minutes. These conditions are indicated, in particular, for forming the B, A3′, A5′, E3′, E5′ spliceosome complexes. Other complexes require only 5 or 15 minutes incubation. For example, for A/B complexes, incubation can be conducted for 10 minutes at about 30° C.

For assembly of the A, H and E (including E3′ and E5′) spliceosome complexes, nuclear extract is preferably first depleted of ATP, as described, e.g., in Michaud and Reed (1991) Genes & Dev. 5:2534, and complex assembly reactions lacked ATP, MgCl₂ and creatine phosphate. For forming and E complex, incubations can be conducted at about 30° C. for about 25-30 minutes. H complexes can also be formed by incubation for 1 or 5 minutes at about 30° C. or for about 5 minutes at 0° C. (see, e.g., Bennett et al., supra). Generally the following times provide the following complexes: 5 minutes for A complex formation, 15 minutes for B complex, 40 minutes for C complex, and 90 minutes plus oligo treatment of another 30 min for spliced mRNP.

In an illustrative embodiment, a spliceosome complex is assembled on an RNA affinity substrate as follows. 20 ng of ³²P labeled pre-mRNA was incubated with 1 μl 12.5 mM ATP; 1 μl 80 mM MgCl₂ ; 1 μl 0.5M Creatine phosphate (diTris salt; Sigma P-4635); 7.5 μl splicing dilution buffer (20 mM HEPES, pH 7.9; 100 mM KCl); 7.5 μl nuclear extract; and a number of μl of water to bring the final volume to 25 μl. This volume can be scaled up 96 fold. The reaction is incubated for 30° C. for 20 minutes or as desired. When one desires to isolate the spliced RNA, the following steps can be taken: 70 μl water are then added to the reaction and 100 μl 2×PK buffer (20 ml IM Tris, pH 8.0; 5 ml 0.5 M EDTA; 6 ml 5M NaCl; 10 ml 20% SDS and bring the volume to 100 ml with water) are added. 5 μl 10 mg/ml Proteinase K is added and the reaction incubated for 10 minutes at 37° C. The reaction is phenol extracted, 2.5 μl glycogen are added to the aqueous phase, vortexed, 600 μl EtOH are added, vortexed and the solution centrifuged for 10-15 minutes. All the liquid is removed. For visualization of the RNA, 6 μl formamide loading dye is added, the mixture is vortexed, boiled, vortexed and centrifuged. 2 μl are loaded on a 6.5, 8, or 15% denaturing polyacrylamide gel at 15 mAmps.

Following formation of a ribonucleoprotein complex on the RNA affinity substrate, the reaction mixture can be subjected to chromatographic separation. This step preferably includes desatling. The chromatographic separation may be gel filtration step or any other chromatographic isolation method, such as an ion exchange chromatographic method. Chromatographic methods are described, e.g., in Robert K. Scopes “Protein Purification: Principles and Practice” Third Edition, 1994, Springer Verlag. In a preferred embodiment, e.g., when the ribonucleoprotein is a spliceosome, the chromatographic step includes gel filtration, such as on Sephacryl S-500 columns equilibrated, e.g., in FSP buffer (20 mM Tris (pH 7.8), 0.1% Triton X-100, 60 mM KCl, 2.5 mM EDTA), loaded and eluted, e.g., as described in Abmayr et al. (1988) PNAS 85:7216 and Reed et al. (1988) Cell 53:949). Different types of spliceosomes elute in different fractions, as described, e.g., in Michaud and Reed (1991), supra and in Bennett et al. (1992), supra.

Following the chromatographic separation, ribonucleoprotein complexes are affinity selected on a matrix that binds directly or indirectly to the affinity tag in the RNA affinity substrate. In a preferred embodiment, the method provides that a low ionic strength is used in passing the ribonucleoprotein complex through the affinity selection step. The low ionic strength buffer may contain, for example, a final sodium chloride concentration of less than about 60 to 100 mM. Preferably, the low ionic strength affinity selection step utilizes a maltose binding protein fused to a sequence specific RNA binding protein which binds the RNA sequence of the RNA affinity tag present in the RNA affinity substrate. In such embodiments, the ribonucleoprotein-RNA affinity substrate complexes are incubated with amylose beads and rotated for about 4 hours at about 4° C. The beads can then be washed and the ribonucleoprotein-RNA affinity substrates eluted using about 12 mM maltose, 20 mM Hepes, pH 7.9, 60 mM NaCl, 10 mM β-mercaptoethanol, and 1 mM PMSF. A person of skill in the art will recognize that certain variations can be introduced in these conditions without significantly affecting the recovery of active and pure ribonucleoprotein complexes.

In embodiments in which binding to the affinity matrix is mediated through another protein or molecule, e.g., Ni⁺⁺, binding, washing and elution can be conducted as known in the art and as provided by manufacturers of these reagents. It is preferably to elute at the lowest salt concentration possible.

The affinity matrix resin can be, e.g., agarose or sepharose. The solid surface for conduction the affinity purification is generally beads, however, any form of solid surface can be used, e.g., flat surfaces. The affinity purification can be conducted in batch or in columns. Magnetic beads can also be used.

In embodiments in which the affinity purification step uses an antibody-ligand pair, antibodies can be prepared as known in the art. Molecules, such as proteins, e.g., antibodies can be linked to an affinity matrix according to methods known in the art. For example, a protein can be linked to a solid support using N-hydroxysuccinimide-activated (NHS) activated agarose or sepharose (e.g.,. Affi-gel (BioRad) and Pharmacia Biotech). N-Hydroxysuccinimide-Agarose can also be obtained from Sigma Chemical Co. (St. Louis, Mo.; Cat. # H 3512 or H 8635).

The method of the invention makes available certain isolated ribonucleoprotein complexes in a purified form not previously available. For example, the isolated spliceosome preparation, isolated by the method of the invention is both highly pure and highly active. Purified spliceosome preparations comprise less than about 50% of contaminating biological material, preferably less than about 40%, 30%, 20%, 10%, and most preferably less than about 1% of contaminating biological material. Contaminating biological material can be proteins or nucleic acids, e.g., RNA.

Purified spliceosomes are preferably biologically active, i.e., they are capable of splicing pre-mRNA in vitro. In general, the purified spliceosome preparations can be chased into completely spliced products where at least about 10%, preferably at least about 20%, 50%, 70%, 90% or more than 90% of the pre-mRNA sequences associated with the isolated spliceosome complexes become completely spliced mRNA in a splicing reaction. The isolated spliceosome preparation of the invention characteristically contain quantitative amounts of 17S U2 small ribonucleoprotein (snRNP), including quantitatively associated amounts of the SP3a polypeptide. The spliceosome preparations of the invention include E complex spliceosome preparations and related spliceosomal intermediate complexes. In general the spliceosome complexes of the invention include specific and quantitatively associated amounts of the U2 snRNP. Other spliceosomes comprise Aly (Zhou et al. (2000) Nature 407:401).

Ribonucleoprotein complexes can consist of isolated proteins; recombinantly produced proteins; or a combination of both. The nucleic acid sequences of spliceosome factors are known in the art (see, e.g., Tables 1 and 2 herein).

RNA can be removed from the ribonucleoprotein complexes, e.g., by treatment with protease free Rnase (e.g., from Boehringer Mannheim), e.g., at about 200 μg /ml, and incubated at about 30° C. for about 10 minutes (see, e.g., Bennett et al. (1992), supra). The following buffer can be used for isolating RNA from spliceosomes: 20 mM HEPES pH 7.9; 60 mM NaCl; 0.1% Triton; 0.01% NaN3.

The purity and protein composition of purified ribonucleoprotein complexes can be analyzed, e.g., by electrophoresis, such as two-dimensional electrophoresis (see, e.g., Bennett et al. (1992), supra). Individual proteins can be identified, e.g., by Western blot (see, e.g., Bennett et al. (1992), supra).

3. Description of Nuclear Pre-mRNA Intronic Sequences

The following description of nuclear pre-mRNA intronic sequences is intended to provide further insight to one skilled in the art to devise constructs useful in the RNA affinity substrates of the invention.

Nuclear pre-mRNA splicing proceeds through a lariat intermediate in a two-step reaction. In contrast to the highly conserved structural elements that reside within group II introns, however, the only conserved features of nuclear pre-mRNA introns are restricted to short regions at or near the splice junctions. For instance, in yeast motifs are (i) a conserved hexanucleotide at the 5′ splice, (ii) an invariant heptanucleotide, the UACUAAC box, surrounding the branch point A (underlined), and (iii) a generally conserved enrichment for pyrimidine residues adjacent to an invariant AG dinucleotide at the 3′ splice site.

Two other characteristics of nuclear pre-mRNA splicing in vitro that distinguish it from autocatalytic splicing are the dependence on added cell-free extracts and the requirement for adenosine triphosphate (ATP). Once in vitro systems had been established for mammalian and yeast pre-mRNA splicing, it was found that a group of trans-acting factors, predominately made up of small nuclear ribonucleoprotein particles (snRNP's) containing U1, U2, U4, U5 and U6 RNA's was essential to the splicing process. Together with the discovery of autocatalytic introns, the demonstration that snRNAs were essential, trans-acting components of the spliceosome argued strongly that group II self-splicing and nuclear pre-mRNA splicing occurring by fundamentally equivalent mechanisms. According to this view, the snRNAs compensate for the low information content of nuclear introns and, by the formation of intermolecular RNA-RNA interactions, achieve the catalytic capability inherent in the intramolecular structure of autocatalytic introns.

Consensus sequences of the 5′ splice site and at the branchpoint are recognized by base pairing with the U1 and U2 snRNP's, respectively. The original proposal that the U1 RNA interacted with the 5′ splice site was based solely on the observed nine-base-pair complementarity between the two mammalian sequences (Rogers et al. (1980) Nature 283:220). This model has since been extensively verified experimentally (reviewed in Steitz et al., in Structure and Function of Major and Minor snRNP Particles, M. L. Bimstiel, Ed. (Springer-Verlag, New York, 1988)). Demonstration of the Watson-Crick interactions between these RNAs was provided by the construction of compensatory base pair changes in mammalian cells (Zhuang et al. (1986) Cell 46:827). Subsequently, suppressor mutations were used to prove the interaction between U1 and 5′ splice site in yeast (Seraphin et al. (1988) EMBO J. 7:2533).

The base pairing interaction between U2 and sequences surrounding the branchpoint was first tested in yeast (Parker et al. (1987) Cell 49:229), where the strict conservation of the branchpoint sequence readily revealed the potential for complementarity. The branchpoint nucleotide, which carries out nucleophilic attack on the 5′ splice site, is thought to be unpaired, and is analogous to the residue that bulges out of an intramolecular helix in domain 6 of group II introns. The base pairing interaction between U2 and the intron has also been demonstrated genetically in mammalian systems (Zhaung et al. (1989) Genes Dev. 3:1545). In fact, although mammalian branchpoint sequences are notable for their deviation from a strict consensus, it has been demonstrated that a sequence identical to the invariant core of the yeast consensus, CUAAC is the most preferred (Reed et al. (1989) PNAS 86:2752).

Genetic evidence in yeast suggests that the intron base pairing region at the 5′ end of U1 RNA per se is not sufficient to specify the site of 5′ cleavage. Mutation of the invariant G at position 5 of the 5′ splice site not only depresses cleavage efficiency at the normal GU site but activates cleavage nearby; the precise location of the aberrant site varies depending on the surrounding context (Jacquier et al. (1985) Cell 43:423; Parker et al. (1985) Cell 41:107; and Fouser et al. (1986) Cell 45:81). Introduction of a U1 RNA, the sequence of which has been changed to restore base pairing capability at position 5, does not depress the abnormal cleavage event; it enhances the cleavage at both wild-type and aberrant sites. These results indicate that the complementarity between U1 and the intron is important for recognition of the splice-site region but does not determine the specific site of bond cleavage (Seraphin et al. (1988) Genes Dev. 2:125; and Seraphin et al. (1990) Cell 63:619).

With regard to snRNPs, genetic experiments in yeast have revealed that the U5 snRNP is an excellent candidate for a trans-acting factor that functions in collaboration with U1 to bring the splice sites together in the spliceosome. U5 is involved in the fidelity of the first and the second cleavage-ligation reactions. For example, a number of U5 mutants exhibit a distinct spectrum of 5′ splice-site usage; point mutations with the invariant nine-nucleotide loop sequence (GCCUUUUAC) in U5 RNA allows use of novel 5′ splice sites when the normal 5′ splice site was mutated. For instance, splicing of detective introns was restored when positions 5 or 6 of the invariant U5 loop were mutated so that they were complementary to the nucleotides at positions 2 and 3 upstream of the novel 5′ splice site when the normal 5′ splice site was mutated. For instance, splicing of defective introns was restored when positions 5 or 6 of the invariant U5 loop were mutated so that they were complementary to the nucleotides at positions 2 and 3 upstream of the novel 5′ splice site. Likewise, mutational analysis has demonstrated the role of the U5 loop sequence in 3′ splice site activation. For example, transcripts which are defective in splicing due to nucleotide changes in either one of the first two nucleotides of the 3′ exon were subsequently rendered functional by mutations in positions 3 or 4 of the U5 loop sequence which permitted pairing with the mutant 3′ exon. (See Newman et al. (1992) Cell 68:1; and Newman et al. (1991) Cell 65:115). It is suggested that first U1 base pairs with intron nucleotides at the 5′ splice site during assembly of an early complex (also including U2). This complex is joined by a tri-snRNP complex comprising U4, U5 and U6 to form a Holliday-like structure which serves to juxtaposition the 5′ and 3′ splice sites, wherein U1 base pairs with intronic sequences at both splice site. (Steitz et al. (1992) Science 257:888-889).

While each of the U1, U2 and U5 snRNPs appear to be able to recognize consensus signals within the intron, no specific binding sites for the U4-U6 snRNP has been identified. U4 and U6 are well conserved in length between yeast and mammals and are found base paired to one another in a simple snRNP (Siliciano et al. (1987) Cell 50:585). The interaction between U4 and U6 is markedly destablized specifically at a late stage in spliceosome assembly, before the first nucleolytic step of the reaction (Pikienly et al. (1986) Nature 324:341; and Cheng et al. (1987) Genes Dev. 1:1014). This temporal correlation, together with an unusual size and sequence conservation of U6, has lead to the understanding that the unwinding of U4 and U6 activates U6 for participation in catalysis. In this view, U4 would function as an antisense negative regulator, sequestering U6 in an inert conformation until it is appropriate to act (Guthrie et al. (1988) Annu Rev. Genet. 22:387). Mutational studies demonstrate a functional role for U6 residues in the U4-U6 interaction domain in addition to base pairing (Vanken et al. (1990) EMBO J 9:3397; and Madhani et al. (1990) Genes Dev. 4:2264).

Mutational analysis of the splicesomal RNAs has revealed a tolerance of substitutions or, in some cases, deletion, even of phylogentically conserved residues (Shuster et al. (1988) Cell 55:41; Pan et al. (1989) Genes Dev. 3:1887; Liao et al. (1990) Genes Dev. 4:1766; and Jones et al. (1990) EMBO J 9:2555). For example, extensive mutagenesis of yeast U6 has been carried out, including assaying the function of a mutated RNA with an in vitro reconstitution system (Fabrizo et al. (1990) Science 250:404), and transforming a mutagenized U6 gene into yeast and identifying mutants by their in vivo phenotype (Madhani et al. (1990) Genes Dev. 4:2264). Whereas most mutations in U6 have little or no functional consequence (even when conserved residues were altered), two regions that are particularly sensitive to nucleotide changes were identified: a short sequence in stem I (CAGC) that is interrupted by the S. prombe intron, and a second, six-nucleotide region (ACAGAG) upstream of stem I.

As described above for group II introns, exonic sequences derived from separate RNA transcripts can be joined in a trans-splicing process utilizing nuclear pre-mRNA intron fragments (Konarska et al. (1985) Cell 42:165-171; and Solnick (1985) Cell 42:157-164). In the trans-splicing reactions, an RNA molecule, comprising an exon and a 3′ flanking intron sequences which includes a 5′ splice site, is mixed with an RNA molecule comprising an exon and 5′ flanking intronic sequences, including a 3′ splice site, and a branch acceptor site. Upon incubation of the two types of transcripts (e.g. in a cell-free splicing system), the exonic sequences can be accurately ligated. In a preferred embodiment the two transcripts contain complementary sequences which allow basepairing of the discontinuous intron fragments. Such a construct can result in a greater splicing efficiency relative to a scheme in which no complementary sequences are provided to potentiate complementation of the discontinuous intron fragments.

The exon ligation reaction mediated by nuclear pre-mRNA intronic sequences can be carried out in a cell-free splicing system. For example, combinatorial exon constructs can be mixed in a buffer comprising 25 mM creatine phosphate, 1 mM ATP, 10 mM MgCl2, and a nuclear extract containing appropriate factors to facilitate ligation of the exons (Konarska et al. (1985) Nature 313:552-557;Krainer et al. (1984) Cell 36:993-1005; and Dignam et al. (1983) Nuc. Acid Res 11:1475-1489). The nuclear extract can be substituted with partially purified spliceosomes capable of carrying out the two transesterification reactions in the presence of complementing extracts. Such spliceosomal complexes have been obtained by gradiant sedimentation (Grabowski et al. (1985) Cell 42:345-353; and Lin et al. (1987) Genes Dev. 1:7-18), gel filtration chromatography (Abmayr et al. (1988) PNAS 85:7216-7220; and Reed et al. (1988) Cell 53:949-961), and polyvinyl alcohol precipitation (Parent et al. (1989) J. Mol. Biol. 209:379-392). In one embodiment, the spliceosomes are activated for removal of nuclear pre-mRNA introns by the addition of two purified yeast “pre-mRNA processing” proteins, PRP2 and PRP16 (Kim et al. (1993) PNAS 90:888-892; Yean et al. (1991) Mol. Cell Biol. 11:5571-5577; and Schwer et al. (1991) Nature 349:494-499).

4. Uses

The methods and compositions of the invention can be used for diagnostic purposes. For example, they can be used to determine whether a subject has an abnormality in the formation of a ribonucleoprotein complex, such as a spliceosome. In one embodiment, the diagnostic method includes obtaining a sample of cells from a subject, e.g., a blood sample or peripheral nuclear mononuclear cells (PBMCs). Such samples can be obtained according to methods known in the art. Ribonucleoprotein complexes can then be formed in vitro from a nuclear extract of the cells from the subject, as described herein. The ribonucleoprotein assembly sequence of the RNA affinity substrate will depend on the particular ribonucleoprotein to be detected. Following the formation of the complex, the presence or absence of certain factors normally present in such complexes can be evaluated. In a preferred embodiment, a ribonucleoprotein complex is first purified, e.g., according to methods described herein, and then the presence or absence of one or more ribonucleoproteins is determined. This can be performed by various methods. In one method, an antibody specific to a ribonucleoprotein is used to determine the presence and/or amount of the protein according to methods well known in the art. Antibodies may be available commercially, or they may be prepared according to methods known in the art. In another embodiment, the presence and/or level of one or more proteins is determined by visualizing the proteins, such as by electrophoresis. For example, a two dimensional electrophoresis can be performed, e.g., as described herein. The comparison of the two dimensional electrophoresis results obtained with ribonucleoprotein complexes of a subject and those of a ribonucleoprotein complex that is known to have all proteins in normal amounts, e.g., a functional ribonucleoprotein, will indicate any differences in composition of the ribonucleoprotein of a subject relative to a normal composition. In yet another embodiment, the composition of a ribonucleoprotein complex is determined using microarrays comprising markers of one or more proteins of the ribonucleoprotein complex. The preparation of microarrays is known in the art. In other methods, one or more proteins can be analyzed to determine the presence of a difference in amino acid sequence relative to a reference, i.e., normal protein. This can be performed, e.g., by using antibodies that specifically recognize mutated forms of these proteins. Alternatively, this can be performed by sequencing at least part of the proteins, e.g., as described herein.

In the case of a diagnostic assay analyzing the composition of spliceosomes, the assay may include analyzing the composition of one or more types of spliceosomes, e.g., type A or E. Other assays may involve preparing a mixture of different types of spliceosomes, e.g., as described in the Examples and analyze essentially all proteins associated with pre-mRNA splicing. In a preferred embodiment, the presence of one or more proteins listed in Tables 1 and/or 2 in spliceosome complexes of a subject is determined. The presence of an abnormal amount, e.g., the absence of one or more proteins listed in Tables 1 and 2 in spliceosomes of a subject is indicative of an abnormality in the spliceosomes, and thus, that the subject is likely to have or to develop a disease associated with abnormal spliceosomes.

Depending on the type of ribonucleoprotein to be characterized, an appropriate substrate can be chosen. For analysis of spliceosomes, one may use pAdL or Ftz pre-mRNA, for example.

Diagnostic methods may also include determining whether the ribonucleoprotein complexes of a subject are functional. This can be done by, e.g., analyzing the RNA that is associated with the ribonucleoprotein complex after purification of the complex, e.g., as described herein. For example, in situations in which the ribonucleoprotein to be analyzed is a spliceosome, an analysis of the RNA substrate following complex purification will reveal whether splicing has occurred. Indeed, if the spliceosomes are functional, a pre-mRNA substrate is spliced into a mature RNA (i.e., the intron was spliced out) during the purification process. In one embodiment, the length of the RNA substrate included in the assay is compared with the length of the RNA obtained after isolation of spliceosome complexes.

The diagnostic assays of the invention are amenable to high throughput diagnostic assays. For example, at least 5, at least 10, 25, 50, 96 or at least 100 samples from subjects can be tested simultaneously, e.g., using robots. In another embodiment, a sample of a single subject is used for testing the functionality and/or presence of one or more ribonucleoproteins.

It is estimated that about 15% of genetic diseases are associated with plicing mutations. These diseases can be directly linked to an abnormality in spliceosomes. Accordingly, the invention can be used for diagnosing numerous conditions. Set forth below are exemplary diseases which can be diagnosed, and optionally treated, according to the invention. In one embodiment, the disease is characterized by photoreceptor degeneration, e.g., Retinitis Pigmentosa. Indeed, mutations in a gene (PRPF31) homologous to Saccharomyces cerevisiae pre-mRNA splicing gene PRP31 was found in families with autosomal dominant Retinitis Pigmentosa linked to chromosome 19q13.4 (RP11; MIM 600138) (Vithana et al. (2001) Mol Cell 8:375). This protein was identified as a spliceosome protein (see Examples). Another protein identified in the Examples as being associated with spliceosomes, i.e, hPrp3 (or U4/U6-90K) was also recently found to be associated with Retinitis Pigmentosa (Hu. Mol. Genetics 11:87 (2002)).

Spinal muscular atrophy (SMA) is also associated with defective ribonucleoprotein complexes. Survival of motor neurons (SMN) protein interacts with spliceosomal snRNP proteins and is critical for snRNP assembly in the cytoplasm. Inhibition of this interaction results in inhibition of pre-mRNA splicing (Pellizonni et al. (1998) Cell 95:615). Low levels of functional SMN results in SMA, which is a neurodegenerative disease of spinal motor neurons. SMN is an essential U snRNP assembly factor and there is a direct correlation between defects in the biogenesis of U snRNPs and SMA (Buhler et al. Hum Mol Genet (1999) 8:2351).

Ribonucleoprotein complexes appear to be involved in rheumatic autoimmune diseases such as systemic lupus erythematosus (SLE), progressive systemic sclerosis, polymyositis, mixed connective tissue disease (MCTD), Sjögren syndrome (SS), and rheumatoid arthritis (RA). These diseases are characterized by the occurrence of autoantibodies to intracellular antigens which are components of large ribonucleoprotein complexes, such as the ribosome and the spliceosome (von Mühlen, C. A., and E. M. Tan (1995) Semin. Arthritis Rheum. 24: 323 and Peng et al. (1997) Antinuclear antibodies. In Textbook of Rheumatology. W. N. Kelley, E. D. Harris, S. Ruddy, and C. B. Sledge, editors. W. B. Saunders Company, Philadelphia, Pa. 250-266 and van Venrooij, W., and G. J. M. Pruijn (1995) Curr. Opin. Immunol. 7: 819). For example, autoantibodies to the Sm antigen are highly specific for SLE, autoantibodies to topoisomerase (anti-Sc170) are exclusively detected in patients with progressive systemic sclerosis, and autoantibodies to tRNA synthetases (e.g., anti-Jo1) occur only in patients with poly- or dermatomyositis (Arbuckle et al. (1998) J Autoimmun 11:431). 20-40% of patients with rheumatoid arthritis (RA), SLE, and mixed connective tissue disease (MCTD) have anti-A2/RA33 autoantibodies, which are directed to the A2 protein of the heterogeneous nuclear ribonucleoprotein complex (hnRNP-A2), an abundant nuclear protein associated with the spliceosome (Skriner et al. (1997) J Clin 100:127. These patients may also have anti-A1 autoantibodies, which are directed to the hnRNP proteins A1 and A1b. In SLE, anti-hnRNP-A/B antibodies frequently occur together with antibodies to two other spliceosome-associated antigens, U1 small nuclear RNP (U1-snRNP) and Sm (Steiner et al. (1996) Int Arch Allergy Immunol 111:314).

Other diseases include fragile X chromosome. In another embodiment, the disease is familial dysautonomia (FD), such as Riley-Day syndrome (see, e.g., Luzzi et al. (1983) Riv Patol Nerv Ment. 104:229. Familial dysautonomia (FD; also known as “Riley-Day syndrome”), an Ashkenazi Jewish disorder, is the best known and most frequent of a group of congenital sensory neuropathies and is characterized by widespread sensory and variable autonomic dysfunction

The methods of the invention can be used to identify yet other diseases associated with abnormal ribonucleoprotein complexes. For example, ribonucleoprotein complexes can be analyzed in subjects having a particular disease, as described herein, in particular those having splicing dysfunctions.

The invention also provides methods for correcting or “normalizing” a ribonucleoprotein abnormality in a subject. For example, a subject lacking a particular ribonucleoprotein can be treated by administering to the subject the particular ribonucleoprotein or a nucleic acid encoding the particular ribonucleoprotein. Proteins or derivatives thereof can be administered to a subject via liposomes. Cellular uptake of proteins may be enhanced by linking a polypeptide sequence enhancing cellular uptake to the protein. For example, a transcytosis peptide, e.g., human immunodeficiency virus (HIV) Tat protein or the antennapedia protein can be linked to the protein. Nucleic acids can be administered in the form of an expression vector, as known in the art. Proteins and nucleic acids can be targeted to particular sites in a subject, e.g., by packaging them in a vector that contains molecules that provide target site recognition.

In other embodiments, the level of a protein is increased by stimulating expression of the gene.

A subject having been identified as overexpressing a particular ribonuclear protein can be treated by the administration of a drug that reduces expression or translation of the protein, e.g., antisense RNA, siRNAs, ribozymes, antibodies, or compounds blocking expression of the gene.

Proteins for administering to a subject in need thereof can be prepared recombinantly, according to methods known in the art, or by purification from a ribonucleoprotein complex obtained, e.g., as described herein.

In certain embodiments, the method of the invention can be used to facilitate in vitro intron-mediated recombinant techniques, such as those described in U.S. Pat. Nos. 6,150,141, 5,780,272 and 5,498,531. In one embodiment of the present invention, the purified splicosomal complexes are used to direct transplicing of exonic units to generate random libraries of shuffled exonic units or to direct assembly of a predetermined sequence of exons. In this combinatorial method, the intronic sequences which flank each of the exon modules are chosen such that gene assembly occurs in vitro through ligation of the exons, mediated by a trans-splicing mechanism. Conceptually, processing of the exons resembles that of a fragmented cis-splicing reaction, though a distinguishing feature of trans-splicing versus cis-splicing is that substrates of the reaction are unlinked. As described above, breaks in the intron sequence can be introduced without abrogating splicing, indicating that coordinated interactions between different portions of a functional intron need not depend on a covalent linkage between those portions to reconstitute a functionally-active splicing structure. Rather, the joining of independently transcribed coding sequences results from interactions between fragmented intronic RNA pieces, with each of the separate precursors contributing to a functional trans-splicing core structure.

The trans-splicing system provides an active set of reagents for trans-splicing wherein the flanking intronic sequences can interact to form a reactive complex which promotes the transesterification reactions necessary to cause the ligation of discontinuous exons. In one embodiment, the exons are flanked by portions of one of a group II intron, such that the interaction of the flanking intronic sequences is sufficient to form functional splicing complexes with involvement of at least one trans-acting factor. For example, the additional trans-acting factor may compensate for structural defects of a complex formed solely by the flanking introns. As described above, domain 5 of the group II intron class can be removed from the flanking intronic sequences, and added instead as a trans-acting RNA element. Similarly, when nuclear pre-mRNA intron fragments are utilized to generate the flanking sequences, the ligation of the exons requires the addition of snRNPs to form a productive splicing complex.

In an illustrative embodiment, the present combinatorial approach can make use of group II intronic sequences to mediate trans-splicing of exons. For example, internal exons can be generated which include domains 5 and 6 at their 5′ end, and domains 1-3 at their 3′ end. The nomenclature of such a construct is (IVS5,6) Exon(IVS 1-3), representing the intron fragments and their orientation with respect to the exon. Terminal exons are likewise constructed to be able to participate in trans-splicing, but at only one end of the exon. A 5′ terminal exon, in the illustrated group II system, is one which is flanked by domains 1-3 at its 3′ end [Exons(IVS1-3)] and is therefore limited to addition of further exonic sequences only at that end; and a 3′ terminal exon is flanked by intron sequences (domains 5 and 6) at only its 5′ end [(IVS5,6)Exon]. Under conditions which favor trans-splicing, the flanking intron sequences at the 5′ end of one exon and the 3′ end of another exon will associate to form a functionally active complex by intermolecular complementation and ligate the two exons together. Such trans-splicing reactions can link the 5′ terminal exon directly to the 3′ terminal exon, or alternatively can insert one or more internal exons between the two terminal exons.

In some cases, trans-splicing reactions by intron-flanked internal exons may be inhibited by a competing inverse-splicing reaction that such internal exons can undergo. Intron-flanked internal exons can participate in intramolecular “inverse-splicing” reactions in which the 3′ end of the exon is spliced to its own 5′ end, so that the exon is circularized (and the intronic sequences are released as a Y-branched ribozyme). Because inverse-splicing is an intramolecular reaction, it can sometimes compete effectively with any trans-splicing reactions, so that few trans-splicing products are produced. In such cases, the inverse-splicing reaction can be inhibited by provision of an antisense nucleic acid that binds to one or the other of the flanking intronic elements. Of course, the antisense nucleic acid will also block one of the trans-splicing reactions that would otherwise be available to the internal exon. Accordingly, use of antisense nucleic acids to control inverse-splicing also limits trans-splicing experiments to a series of sequential reactions—a sequential trans-splicing reaction according to the present invention.

In another embodiment of the present trans-splicing combinatorial method, the exons, as initially admixed, lack flanking intronic sequences at one or both ends, relying instead on a subsequent addition of flanking intronic fragments to the exons by a reverse-splicing reaction. Addition of the flanking intron sequences, which have been supplemented in the exon mixture, consequently activates an exon for trans-splicing. The reverse-splicing reaction of group II introns can be used to add domains 1-3 to the 3′ end of an exon as well as domains 5-6 to the 5′ end of an exon. The reversal reaction for branch formation can mediate addition of 3′ flanking sequences to an exon. For example, exon modules having 5′ intron fragments (e.g. domains 5-6) can be mixed together with little ligation occurring between exons. These exons are then mixed with a 2′-5′ Y-branched intron resembling the lariat-IVS, except that the lariat is discontinuous between domains 3 and 5. The reverse-splicing is initiated by binding of the IBS 1 of the 5′ exon to the EBS 1 of the Y-branched intron, followed by nucleophilic attack by the 3′-OH of the exon on the 2′-5′ phosphodiester bond of the branch site. This reaction results in the reconstitution of the 5′ splice-site with a flanking intron fragment comprising domains 1-3.

Addition of intronic fragments by reverse-splicing and the subsequent activation of the exons presents a number of control advantages. For instance, the IBS:EBS interaction can be manipulated such that a variegated population of exons is heterologous with respect to intron binding sequences (e.g. one particular species of exon has a different IBS relative to other exons in the population). Thus, sequential addition of intronic RNA having discrete EBS sequences can reduce the construction of a gene to non-random or only semi-random assembly of the exons by sequentially activating only particular combinatorial units in the mixture. Another advantage derives from being able to store exons as part of a library without self-splicing occurring at any significant rate during storage. Until the exons are activated for trans-splicing by addition of the intronic sequences to one or both ends, the exons can be maintained together in an effectively inert state.

When the interactions of the flanking introns are random, the order and composition of the internal exons of the combinatorial gene library generated is also random. For instance, where the variegated population of exons used to generate the combinatorial genes comprises N different internal exons, random trans-splicing of the internal exons can result in N<y> different genes having y internal exons. Where 5 different internal exons are used (N=5) but only constructs having one exon ligated between the terminal exons are considered (i.e. y=1) the present combinatorial approach can produce 5 different genes. However, where y=6, the combinatorial approach can give rise to 15,625 different genes having 6 internal exons, and 19,530 different genes having from 1 to 6 internal exons (e.g. N<1>+N<2> . . . +N<y−1>+N<y>. It will be appreciated that the frequency of occurrence of a particular exonic sequence in the combinatorial library may also be influenced by, for example, varying the concentration of that exon relative to other exons present, or altering the flanking intronic sequences of that exon to either diminish or enhance its trans-splicing ability relative to the other exons being admixed.

However, the present trans-splicing method can be utilized for ordered gene assembly, and carried out in much the same fashion as automated oligonucleotide or polypeptide synthesis. For example, mammalian pre-mRNA introns are used to flank the exon sequences, and splicing is catalyzed by addition by splicing extract isolated from mammalian cells. The steps outlined can be carried out manually, but are amenable to automation. The 5′ terminal exon sequence is directly followed by a 5′ portion of an intron that begins with a 5′ splice-site consensus sequence, but does not include the branch acceptor sequence. The flanking intron fragment further includes an added nucleotide sequence at the 3′ end of the downstream flanking intron fragment. The 5′ end of this terminal combinatorial unit is covalently linked to a solid support. For example, exon 2 is covalently joined to exon 1 by trans-splicing. The internal shuffling unit that contains exon 2 is flanked at both ends by intronic fragments. Downstream of exon 2 are intron sequences similar to those downstream of exon 1, with the exception that in place of sequence A the intronic fragment of exon 2 has an added sequence B that is unique, relative to sequence A. Exon 2 is also preceded by a sequence complementary to A (designated A′), followed by the nuclear pre-mRNA intron sequences that were not included downstream of exon 1, including the branch acceptor sequence and 3′ splice-site consensus sequence AG.

Transplicing may require the complementation of purified spliceosome complexes with factors which are involved early on in the splicing process.

5.Kits

The invention further provides kits for use, e.g., in purifying ribonucleoprotein complexes, such as spliceosomal complexes. Kits may comprise one or more of: an RNA affinity substrate; a fusion protein comprising an affinity tag binding polypeptide and a ligand binding polypeptide; chromatographic separation reagents; and affinity purification reagents. Kits can be used, e.g., for diagnostic purposes, such as for determining the presence of abnormal ribonucleoprotein complexes in a subject. Other kits may comprise reagents for in vitro splicing reactions, e.g., isolated ribonucleoprotein complexes or fractions thereof. The reagents can be packaged in a suitable container. The kit can further comprise instructions for using the kit to purify a particular ribonucleoprotein complex or a complex selected by the user.

The present invention is further illustrated by the following examples, which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory);, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

In the current model for spliceosome assembly, U1 snRNP binds to the 5′ splice site in the ATP-independent E complex followed by U2 snRNP binding to the branchpoint sequence (BPS) in the ATP-dependent A complex. Surprisingly, we find that highly purified E complex contains both U1 and U2 snRNPs, including the U2 snRNP-associated factors SF3a and SF3b. Pre-mRNA in purified E complex is chased into spliced products in extracts lacking SF3a, and SF3a is essential for E complex assembly. The BPS is not required for association of U2 snRNP with the E complex, indicating that U2-BPS base-pairing is established in the A complex. These data suggest a new model for spliceosome assembly in which U1 and U2 snRNPs associate in the E complex and then an ATP-dependent step results in highly stable binding of U2 snRNP to the BPS in the A complex.

### Example 1

### Isolation and Characterization of Functional Spliceosomes

In previous studies, functional mammalian spliceosomes were partially purified by gel filtration under conditions compatible with splicing (60 mM salt) (Jamison, S. F. et al. (1992) Mol. Cell Biol. 12:4279-87; Michaud, S. et al. (1991) Genes Dev. 5:2534-46; Michaud, S. et al. (1993) Genes Dev. 7:1008-20). In contrast, for determnining protein compositions, complexes were isolated by gel filtration, treated with high salt (250 mM salt) and purified by biotin-avidin affinity selection (Bennett, M. et al. (1992) Genes Dev. 6:1986-2000; Michaud, S. et al (1993) Genes Dev. 7:1008-20; Gozani, O. et al. (1994) EMBO J. 13:3356-67). Because there are significant differences in the compositions of the complexes isolated by these and other methods, we have now characterized the E complex using a recently developed method for isolating spliceosomes that are both highly purified and functional. In this procedure, spliceosomes are assembled on pre-mRNA which is pre-bound to the maltose binding protein (MBP). The spliceosomes are then isolated by gel filtration, bound to amylose beads, and gently eluted with maltose. The resulting MBP-purified spliceosomes are active in splicing when incubated in complementing extracts (see below).

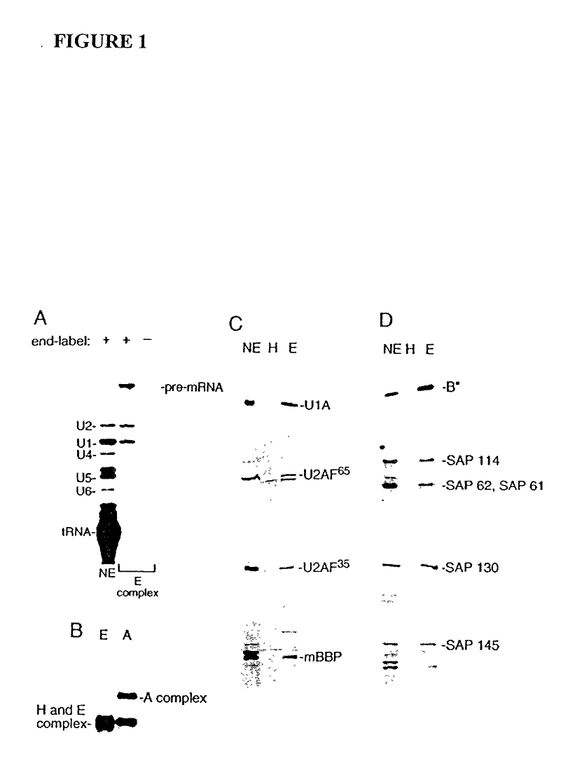

FIG. 1 shows the SnRNA and protein compositions of purified E complex using the new method. (A) SnRNAs in purified E complex. Total RNA was extracted from the E complex (lane 3), end-labeled (lane 2) and fractionated on an 8% polyacrylamide gel. As a marker for the snRNAs, total RNA was extracted from nuclear extract and end-labeled (lane 1). RNAs were visualized by phosphorimager analysis. The low level of U5 snRNA detected in the E complex may be the same as the ATP-independent association of U5 snRNP detected previously (Chabot et al., 1985). The significance of this interaction is not known. (B) Native gel analysis of E and A complexes. ³²P-labeled AdML pre-mRNA was incubated in splicing extracts in the absence (lane 1) or presence of ATP (lane 2), and heparin was added prior to loading onto a 1% agarose gel. The bands corresponding to the H, E and A complexes are indicated. (C) Analysis of proteins in purified E complex. Total protein was prepared from equivalent amounts of purified E and H complexes, separated on a 9% SDS gel, transferred to nitrocellulose and probed with U1A, U2AF⁶⁵, U2AF³⁵, and mBBP antibodies as indicated. The smaller bands detected with the U2AF65 and SAP 145 antibodies may be breakdown products. The extra bands detected in nuclear extract with the mBBP antibody may be other forms of this protein (Arning et al., 1996) (D) Same as C except blots were probed with antibodies to the U2 snRNP components, SF3a, SF3b (SAP 130 and SAP 145) and B″ as indicated.

Significantly, both U1 and U2 snRNAs are detected in the MBP-purified E complex (FIG. 1A). Comparison of these snRNAs by ethidium bromide-staining and end-labeling indicates that they are present in the E complex in about a one to one ratio. The presence of U2 snRNA is not due to contaminating A complex as no A complex is detected in the E complex reactions after heparin-treatment and fractionation on a native agarose gel (FIG. 1B; note that E and H complexes co-migrate under these gel conditions) (Das, R. et al. (1999) RNA 5:1504-08; Michaud, S. et al. (1993) Genes Dev. 7:1008-20).

Western analysis of the MBP-purified E complex revealed the presence of several proteins expected to be in the E complex, including the U1 snRNP protein U1A, both subunits of U2AF, and the branchpoint binding protein, mBBP/SF1 (referred to hereafter as MBBP) (Arning, S. et al. (1996) RNA 2:794-810; Bennett, M. et al. (1992) Genes Dev. 6:1986-2000; Berglund, J. A. et al. (1997) Cell 89:781-87). All of these proteins are specifically associated with the E complex, as they were not detected in the hnRNP complex H (FIG. 1C).

We next asked whether U2 snRNP proteins were present in the MBP-purified E complex. U2 snRNP can be isolated in a 12S and a 17S form (Behrens, S. E. et al. (1993) Proc. Natl. Acad. Sci. USA 90:8229-33; Behrens, S. E. et al. (1993) Mol. Cell Biol. 13:307-19). The B″ protein is a stable component of both forms (Brehrens, S. E. et al. (1993) Proc. Natl. Acad. Sci. USA 90:8229-33). In contrast, the two essential multimeric splicing factors, SF3a and SF3b, are present only in the 17S form (Behrens, S. E. et al. (1993) Proc. Natl. Acad. Sci. USA 90:8229-33); Behrens, S. E. et al. (1993) Mol. Cell Biol. 13:307-19; Brosi, R. et al. (1993) Science 262:102-05;Kramer, A. et al. (1999) J. Cell. Biol. 145:1355-68; Staknis, D. et al. (1994) Mol. Cell Biol. 14). SF3a consists of three subunits (spliceosome-associated proteins (SAPs) 61, 62 and 114), and SF3b consists of four subunits (SAPs 49, 130, 145 and 155) (Brosi, R. et al. (1993) J. Biol. Chem 268:17640-46; Das, B. K. et al. (1999) Mol. Cell Biol. 19:6796-802;Kramer, A. et al. (1999) J. Cell Biol. 145:1355-68).

Significantly, B″, as well as SF3a and SF3b, were detected in the MBP-purified E complex (FIG. 1D, and data not shown; see below for description of the antibody generated against SF3a). None of the U2 snRNP proteins were present in the H complex (FIG. 1D). We conclude that 17S U2 snRNP is specifically associated with the E complex.

To determine whether the 17S U2 snRNP components were quantitatively associated with the E complex or were only present in a subpopulation of this complex, we used a native gel assay to ask whether antibodies to 17S U2 snRNP can supershift the E complex. For comparison, we also examined the A and B complexes which are known to contain 17S U2 snRNP. Agarose gels were used for the assays as these gels were recently shown to resolve the ATP-dependent spliceosomal complexes (A, B, and C), as well as the E and H complexes (Das, R. et al. (1999) RNA 5:1504-08). The E complex is not stable in the presence of heparin whereas the ATP-dependent complexes are heparin-resistant.

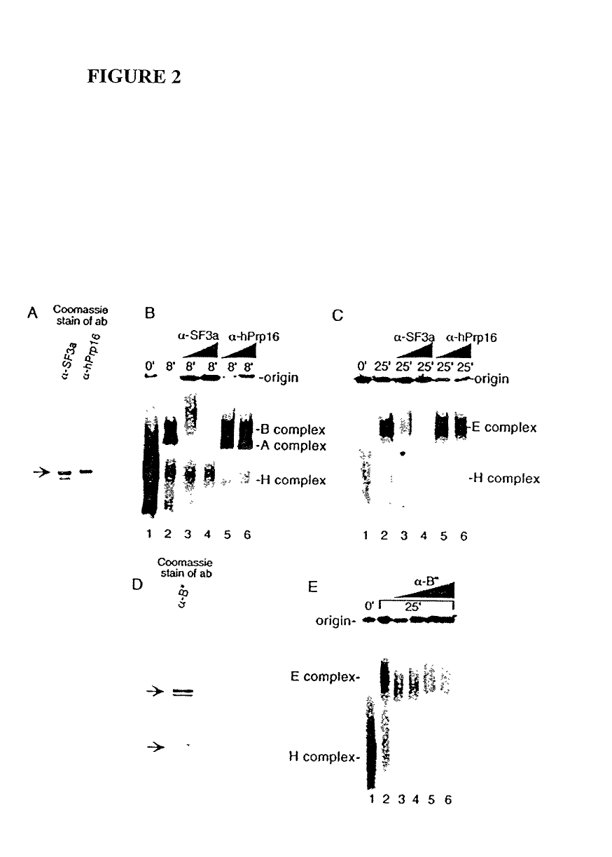

FIG. 2 shows that U2 snRNP is stoichiometrically associated with the E and A complexes. (A) Affinity-purified SF3a and hPrp1 6 antibodies were separated by SDS PAGE. The arrow indicates the antibody heavy chain. (B and C) The A and B spliceosomal complexes were assembled on ³²p-labeled AdML pre-mRNA in presence of ATP, complexes were incubated without antibody (lanes 1 and 2), with SF3a antibody (lanes 3 and 4), or with hPrp16 antibody (lanes 5 and 6) and fractionated on a native agarose gel. The H, A, and B complexes are indicated. The supershift complexes are detected in the well of the gel. (C) Same as B except the E complex was assembled in absence of ATP. The E and H complexes are indicated, and the supershifted complex is detected in the well of the gel. (D) Affinity-purified B″ antibody was separated on by SDS PAGE. The arrows indicate the antibody heavy and light chains. (E) The E complex was assembled on ³²P-labeled AdML pre-mRNA in absence of ATP, and complexes were incubated without (lanes 1 and 2) or with the B″ antibody and fractionated on a native agarose gel.

For the supershift assay, we first tested the SF3a antibody. An antibody to the catalytic step II protein, hPrp16 (Zhou, Z. et al. (1998) EMBO J. 17:2095-106), was used as a negative control. The antibodies were purified under identical conditions and adjusted to equal levels (FIG. 2A). As expected, the A and B complexes were supershifted with the SF3a antibody, but not with an equal amount of the hPrp16 antibody (FIG. 2B). Significantly, the E complex was also efficiently supershifted with the SF3a antibody, but not with the hPrp16 antibody (FIG. 2C). We conclude that SF3a is quantitatively associated with the E complex.

In contrast to SF3a, B″ is very tightly associated with U2 snRNP (Behrens, S. E. et al. (1993) Mol. Cell Biol. 13:307-19). Thus, to determine whether the entire U2 snRNP is likely to be quantitatively associated with the E complex, we carried out the supershift assay using the B″ antibody (FIG. 2D). As shown in FIG. 2E, the E complex is supershifted in a dose-dependent manner by the B″ antibody. These data, together with the results in FIG. 1, indicate that U2 snRNP is specifically and quantitatively associated with the E complex. The presence of U2 snRNP in the E complex is likely to be general, as the SF3a antibody also quantitatively supershifts the E complex assembled on Ftz pre-mRNA.

### Example 2

### US snRNP Associates with the E Complex Independently of the BPS

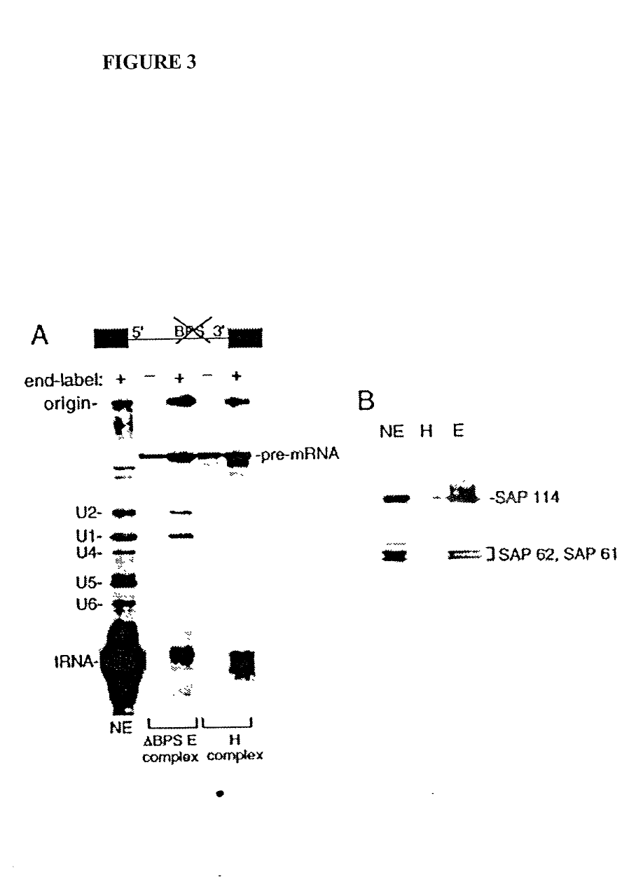

Previous studies have shown that the stable binding of U2 snRNP in the A complex requires the BPS (Champion-Arnaud, P. et al. (1995) Mol. Cell Biol. 15:5750-56; Query, C. C. et al. (1996) EMBO J. 15:1392402; Query, C. C. et al. (1997) Mol. Cell Biol. 17:2944-53). To determine whether the association of U2 snRNP with the E complex is also BPS-dependent, we assembled the E complex on a pre-mRNA lacking the BPS. This mutant is unable to form the A complex, but forms the E complex efficiently (Champion-Arnaud, P. et al. (1995) Mol. Cell Biol. 15:5750-56; Query, C. C. et al. (1996) EMBO J. 15:1392-402). Significantly, both U1 and U2 snRNAs were detected in the MBP-purified ΔBPS E complex (FIG. 3A). Moreover, the 17S form of U2 snRNP is present in the ΔBPS E complex as the subunits of SF3a/b were detected on Western blots of this complex (FIG. 3B and data not shown). We conclude that U2 snRNP is associated with the E complex via a BPS-independent interaction.

FIG. 3 shows that U2 snRNP associates with the E complex in the absence of the BPS. (A) The E and H complexes were assembled on ³²P-labeled ΔdML-M3ΔBPS pre-mRNA and fractionated by gel filtration, affinity-purified by binding to amylose beads and eluted with maltose. Equal amounts of pre-mRNA were prepared from purified E and H complexes, end-labeled with ³²P-pCp and RNA ligase, and fractionated on an 8% polyacrylamide gel. The bands corresponding to pre-mRNA and nuclear RNAs are indicated. (B) Western analysis. Total protein was prepared from equivalent amounts of purified E and H complexes and separated on a 9% SDS gel, transferred to nitrocellulose and probed with the SF3a antibody.

### Example 3

### SF3a is Functional in the E Complex

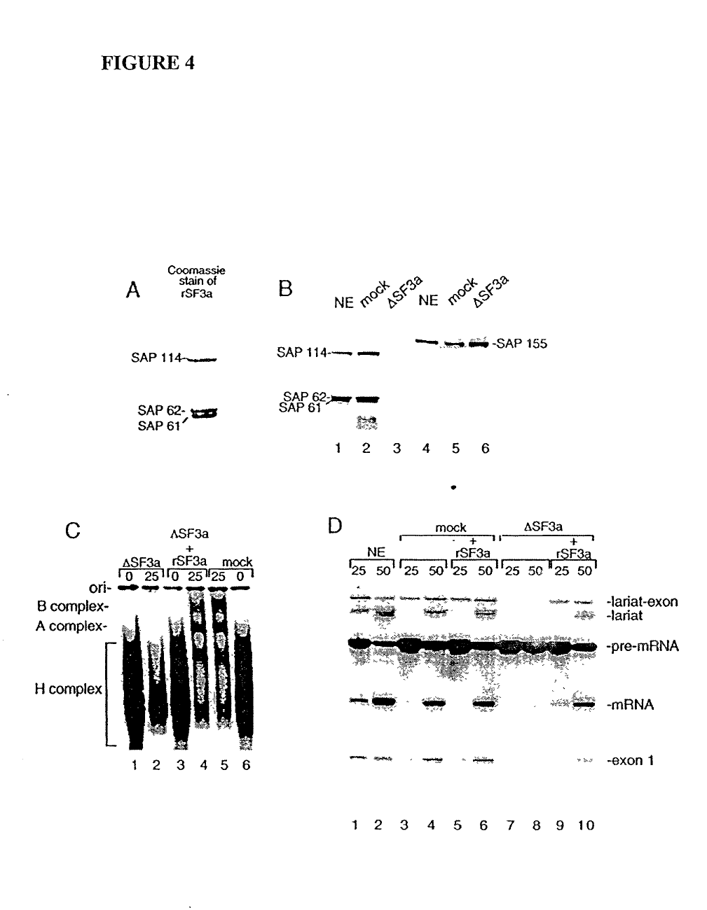

To determine whether U2 snRNP is functionally associated with the E complex, it was first necessary to obtain nuclear extracts specifically lacking U2 snRNP activity. Because this snRNP is so abundant, it is difficult to completely immunodeplete it and, at the same time, retain a highly active extract. Oligonucleotide-directed Rnase H inactivation of U2 snRNA is not sufficient for similar reasons. Thus, as an alternative strategy, we raised a polyclonal antibody to the 17S U2 snRNP-specific SF3a complex, reasoning that an antibody to the entire complex may be sufficiently high-affinity to use for efficient and specific immunodepletions. To raise the antibody, the three recombinant subunits of SF3a were co-expressed in baculovirus. Superose 6 gel filtration revealed that all three proteins were present in a discrete complex in a 1:1:1 stoichiometry (FIG. 4A). Significantly, a rabbit polyclonal antibody raised against the recombinant SF3a (rSF3a) specifically recognizes all three SF3a subunits on a Western blot of total HeLa cell nuclear extract (FIG. 4B, NE).

To determine whether the antibodies could be used to prepare a highly active immunodepleted extract, we carried out immunodepletion/reconstitution assays. Little depletion of SF3a or U2 snRNP was detected in nuclear extract under normal splicing conditions. However, when the salt in the nuclear extract was raised to 700 mM, efficient depletion of SF3a was observed with the SF3a antibody, but not in the mock control (FIG. 4B, lanes 2 and 3). Significantly, other U2 snRNP components, such as SF3b, were not co-depleted (e.g. FIG. 4B, lane 6). To determine whether spliceosome assembly is blocked in the ΔSF3a extract, AdML pre-mRNA was incubated in ΔSF3a or mock-depleted extracts. As shown in FIG. 4C (lanes 1, 2), A and B complex assembly is blocked in the ΔSF3a-depleted, but not in the mock-depleted, extract (lanes 5, 6). Importantly, rSF3a efficiently restores spliceosome assembly in the ΔSF3a extract (lanes 3, 4) and in a dose-dependent manner . We conclude that SF3a can be depleted from nuclear extract and substituted with rSF3a to regain efficient spliceosome assembly. Splicing is also inhibited in the ΔSF3a extract but not in the mock-depleted extract (FIG. 4D, lanes 3, 4 and 7, 8). Moreover, addition of rSF3a efficiently restores splicing (FIG. 4D, lanes 9, 10). Taken together, these data indicate nuclear extracts can be specifically depleted of the essential U2 snRNP component, SF3a, and are highly active when complemented with recombinant SF3a.

FIG. 4 shows SF3a immunodepletion and reconstitution with recombinant SF3a. (A) Coomassie blue-staining of rSF3a complex purified from baculovirus. (B) Western blot of nuclear extract (lanes 1 and 4), mock-depleted extract (lanes 2 and 5) and ASF3a-depleted extract (lanes 3 and 6) probed with SF3a or SAP 155 antibodies as indicated. (C) Immunodepletion/add-back assays of spliceosome assembly. AdML pre-mRNA was incubated in SF3a-depleted (lanes 1-4) or mock-depleted (lanes 5 and 6) extracts for the times indicated. rSF3a (120 ng) was added to the ΔSF3a extract in lanes 3 and 4. Spliceosomal complexes were analyzed on a 2% native agarose gel. Ori indicates the gel origin. (D) Same as C except that splicing products were analyzed on a 13.5% polyacrylamide denaturing gel. Splicing intermediates and products are indicated.

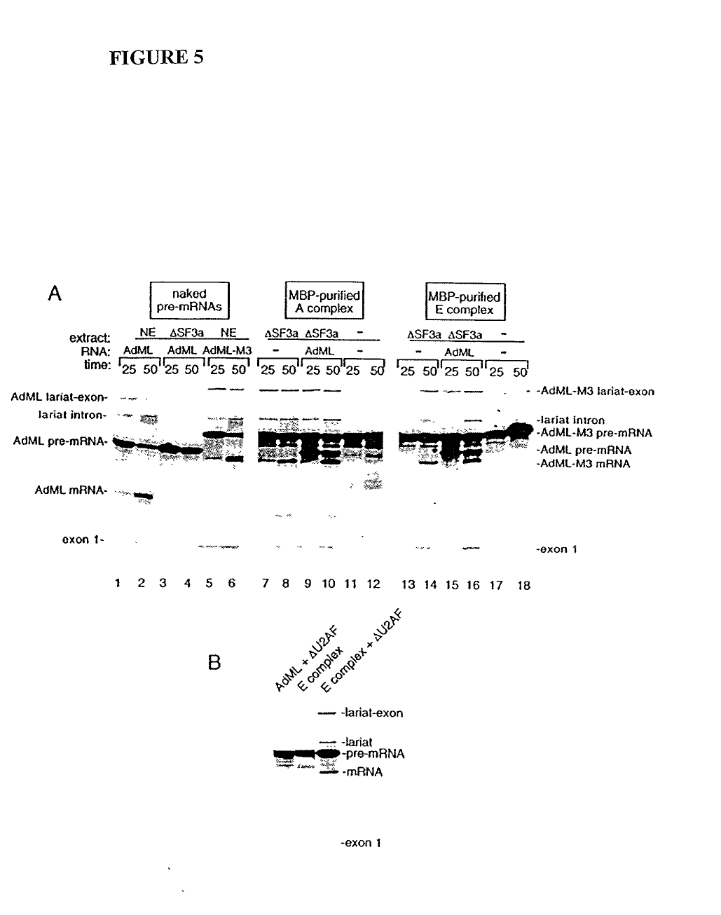

We next asked whether the MBP-purified E complex could be chased to spliced products in the ΔSF3a extract (FIG. 5). MBP-purified A complex, which should contain functional SF3a, was used as a positive control. Both E and A complexes were assembled on AdML-M3 pre-mRNA which contains the 3 hairpins used for the MBP-spliceosome purification. AdML pre-mRNA, which lacks these hairpins, was used as a control in some of the assays (see below). As expected, no splicing was observed when naked AdML pre-mRNA (lanes 3, 4) was incubated in the ΔSF3a extract for 25′ or 50′. Likewise, splicing did not occur when either the purified A complex (lanes 11, 12) or the purified E complex (lanes 17, 18) were incubated under splicing conditions in the absence of extract. In contrast, splicing intermediates and products were detected when the A complex was incubated in the ΔSF3a extract (FIG. 5, lanes 7, 8). Significantly, splicing also occurred when the purified E complex was incubated in the ΔSF3a extract (FIG. 5, lanes 13, 14).

One possible interpretation of these data is that the splicing observed with the purified E and A complexes is due to splicing of the pre-mRNA present in these complexes. Alternatively, the SF3a present in these complexes may simply be complementing the ΔSF3a extract to splice the pre-mRNA. To distinguish between these possibilities, we carried out a mixing experiment using two different AdML derivatives. The purified E and A complexes were assembled on AdML-M3 pre-mRNA which contains a longer second exon than AdML pre-mRNA (see Methods). The products generated from splicing naked AdML or AdML-M3 pre-mRNA in normal nuclear extract are shown in FIG. 5, lanes 1, 2 and 5, 6, respectively. Significantly, efficient splicing of only the AdML-M3 was detected when AdML pre-mRNA was mixed with the purified A complex (lanes 9, 10) or with the purified E complex (lanes 15, 16). This observation indicates that the SF3a in these complexes is not complementing the ΔSF3a extract to splice the naked pre-mRNA. We conclude that SF3a is not only a functional component of the A complex, but also of the E complex.

The purified E complex can also be chased to spliced products in a U2AF-depleted extract (FIG. 5b), indicating that U2AF is a functional component of the E complex. The observation that the pre-mRNA in the E complex is not completely spliced in either the ΔSF3a or ΔU2AF extracts may be because a portion of the complex dissociates during purification.

FIG. 5 shows that SF3a is functionally associated with the purified E complex. (A) ADML pre-mRNA (lanes 1 and 2) or AdML-M3 pre-mRNA (lanes 5 and 6) was incubated under standard splicing conditions in nuclear extract. AdML pre-mRNA was incubated in SF3a-depleted extract (lanes 3 and 4). MBP-purified A complex (lanes 7-12) or E complex (lanes 13-18) were incubated under the indicated conditions. (B) AdML pre-mRNA (lane 1) or affinity-purified E complex (lanes 3) was incubated under splicing conditions in U2AF⁶⁵-depleted extract. Affinity-purified E complex incubated under splicing conditions in the absence of extract is shown in lane 2. Splicing products were separated on 13.5% denaturing polyacrylamide gel. Splicing intermediates and products are indicated.

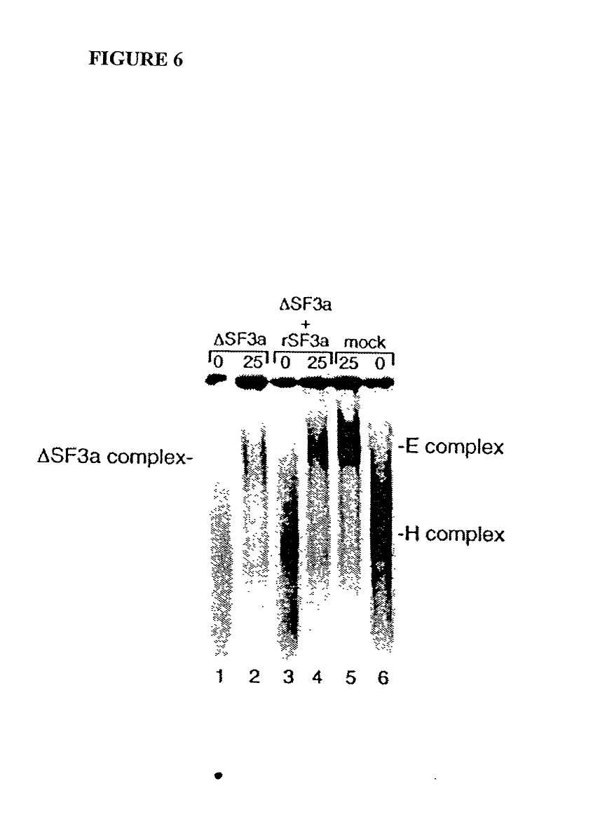

The data presented above indicate that SF3a is a functional component of the E complex. As SF3a is an essential component of 17S U2 snRNP, and this snRNP is present in the purified E complex (FIG. 1), it is likely that the entire U2 snRNP is a functional component of the E complex. To obtain evidence that SF3a (and U2 snRNP) is required for E complex assembly, we investigated complex assembly in the ΔSF3a extract (FIG. 6). When AdML pre-mRNA was incubated in the ΔSF3a extract, the levels of E complex were significantly decreased. In addition, low levels of a complex (designated the ΔSF3a complex), which runs with slightly faster mobility than the E complex, were reproducibly detected (FIG. 6). Significantly, addition of rSF3a to the ΔSF3a extract restores the E complex (FIG. 6). These data indicate that SF3a is required for E complex assembly.

FIG. 6 shows that SF3a is required for E complex assembly. AdML pre-mRNA was incubated in the absence of ATP for the times indicated in SF3a-depleted extract (lanes 1-4) or mock-depleted extract (lanes 5 and 6). rSF3a was added to SF3a-depleted extract in lanes 3 and 4. Reactions were fractionated on a 1.5% native agarose gel. The ΔSF3a complex, and the E and H complexes are indicated.

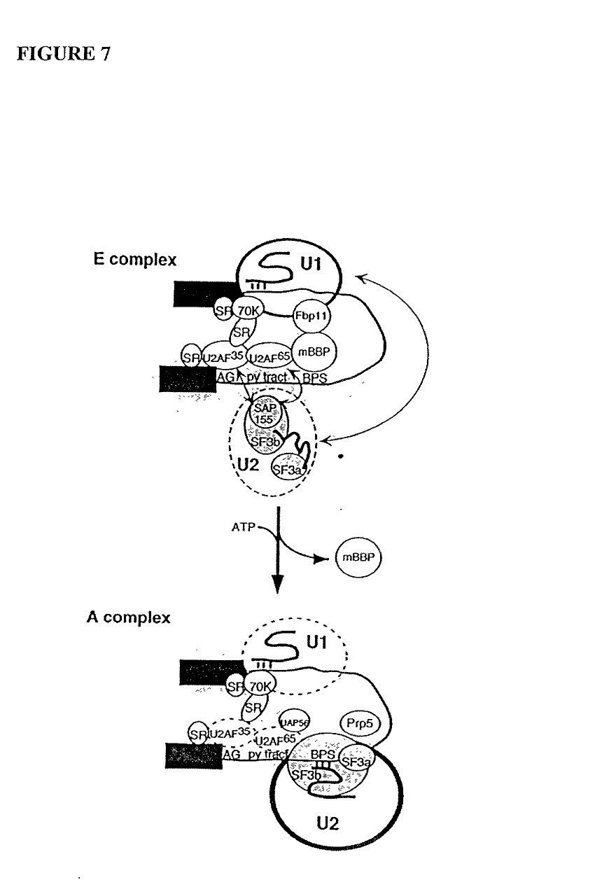

FIG. 7 depicts a model for the early steps in spliceosome assembly. The tight binding of U1 and U2 snRNPs is indicated by the thick-lined circles, and the loose binding of these snRNPs and U2AF is indicated by the dashed circles.

### Example 4

### Materials and Methods

Plasmids The plasmid encoding wild-type AdML pre-mRNA was described in (Michaud, S. et al. (1993) Genes Dev. 7:1008-20). AdML-M3 pre-mRNA contains three phage R17 MS2 binding sites at the 3′ end. AdML-M3ABPS was constructed from AdML-M3 and LUC pre-mRNA which lacks the BPS (Champion-Arnaud, P. et al. (1995) Mol. Cell Biol. 15:5750-56). AdML and AdML-M3 were linearized with Bam HI and Xba I, respectively, for transcription with T7 RNA polymerase.

Isolation and analysis of functional spliceosomal complexes Purification of functional spliceosomal complexes was carried out as follows. An Adenovirus major late pre-mRNA (AdML-M3), which contains three phage R17-MS2 coat protein binding sites at the end of exon 2, was incubated with a, fusion protein consisting of the MS2 coat protein and the maltose binding protein (MBP) in a buffer containing 20 mM Hepes, pH 7.9, 60 mM NaCl. The MS2/MBP fusion protein was expressed in E. coli, and purified by binding to amylose beads according to the manufacturer (NEB). The fusion protein and AdML-M3 pre-mRNA were incubated on ice for 20 minutes, and the binding was assayed on a 1.5% native agarose gel. Spliceosomes were assembled on the MS2/MBP/AdML-M3 complex using standard conditions and isolated by gel filtration (Bennett, M. et al. (1992) Genes Dev. 6:1986-2000). Subsequently, the spliceosomes were affinity-selected on amylose beads by rotating for 4 hrs at 4 degrees and eluted with 12 mM maltose, 20 mM Hepes, pH 7.9, 60 mM NaCl, 10 mM beta-mercaptoethanol, 1 mM PMSF. For assembly of the E and H complexes, nuclear extract was depleted of ATP, and the reactions lacked ATP and MgCl₂ (Michaud, S. et al. (1993) Genes Dev. 7:1008-20) and were incubated at 30° C. for 25 minutes. For A/B complex, pre-mRNA was incubated under standard splicing conditions for 10 minutes at 30° C. For western analysis, total protein was prepared from equivalent amounts of each purified complex, separated by SDS PAGE, and transferred to nitrocellulose. All rabbit antibodies were used at 1:1000 dilution. Tissue culture supernatant from the B″ monoclonal antibody was used undiluted. Secondary antibodies were horseradish peroxidase-linked, and the ECL detection system (Amersham) was used. For identification of snRNAs, total RNA was prepared from equivalent amounts of each purified complex and end labeled with (³²P)pCp and RNA ligase.

Native gel supershift assay SF3a, hPrp16, and B″ antibodies were purified by binding to protein A beads and eluted with Tris-glycine, pH 3. For the supershift assay of E and A/B complexes, splicing extracts (25 μl) were incubated for an additional 15 minutes at room temperature with 480 ng and 960 ng of purified SF3a or hPrp16 antibody. The purified B″ antibody was used at 100, 200, 400 or 600 ng for supershift of the E complex. Complexes were analyzed on native agarose gels as described (Das, R. et al. (1999) RNA 5:1504-08).

Immunodepletion and reconstitution of SF3a Recombinant His-tagged SF3a was produced using a baculovirus expression system (Gibco/BRL). SAPs 61, 62 and 114 were expressed separately initially. SF9 cells were then infected with the three viruses, and after 48 hr of infection, cells were harvested and lysed in 50 mM Tris-HCL (pH 8.5), 10 mM 2-mercaptoethanol, 1 mM PMSF and 1% Triton X-100 at 4° C. The SF3a complex was purified on nickel agarose (Qiagen). Rabbit polyclonal antibodies were raised against the recombinant SF3a complex (Covance Research Products, Denver, Pa.). Immunodepletion of SF3a was carried out according to Zhou and Reed (1998). For reconstitution with recombinant SF3a, 60-120 ng rSF3a were added to 7.5 μl of SF3a-depleted extracts in a 25 μl splicing reaction.

### Example 5

### Purification of Functional Spliceosomes or mRNPs and Identification of Functional Associated Proteins

Spliceosome complexes were formed on two RNA substrates, each having three MS2 binding sites located 3′: one having the Adenovirus Major Late (AdML) pre-mRNA and the other having the Fushi Tarazu (Ftz) pre-mRNA (as described in Zhou et al. (2000) Nature 407:401). Spliceosome complexes were prepared as follows. Substrate RNA-Tag Protein (RTP) complexes were prepared by incubating 10 μl of substrate RNA comprising MS2 binding sites (hairpins) (200 ng/μl) and 30 μl of Maltose binding protein-MS2-Coat-Protein (5 mg/ml) on ice for 1-2 hours; then adding 172 μl of SDB (20 mM HEPES, 100 mM KCl) and incubating on ice for another 20 minutes. The Maltose binding protein-MS2 coat-protein consisted of full-length MBP-LVPRGSH-MRGSHHHHHH-full-length MS2 coat protein (SEQ ID NO: 8). The sequence “LVPRGSH” (SEQ ID NO: 9) is a thrombin cleavage site and “MRGSHHHHHH” (SEQ ID NO: 10) is a 6×His tag. The substrate RNA was prepared as described herein. The RTP complex can be detected on 1.5% agarose gel.

Nuclear extracts were prepared as follows from 50 liters of HeLa cells. The spliceosomes were assembled in the cold room, on ice using cold buffers and cold autoclaved glassware. 50 liters of HeLa cells were pelleted. A small aliquot of the cells was checked for lysis by gently pipetting cells into an eppendorf tube and mixing with an equal volume of trypan blue, and visualization of an aliquot on a slide under a microscope. The cells were brought to 5 packed cell volumes (PCVs) with hypotonic buffer (10 mM HEPES, pH 7.9; 1.5 mM MgCl2; 10 mM KCl; 0.2 mM PMSF; 0.5 mM DTT). The cells were quickly but gently resuspended. The cells were centrifuged for 5 minutes at 3K in a cold HA6000 rotor. The supernatant was poured off, and the cells were brought to 3× the original PCV with hypotonic buffer. The cells were let swell for 10 minutes. The cells were then poured into a cold dounce and dounced slowly and steadily twelve times when 90% of the cells were lysed, as indicated by trypan blue staining. The dounced cells were centrifuiged at 4K for 15 minutes in the orange capped tubes. The pellet contains the nuclei. 1/2 pelleted nuclei volume (PNV) of low salt buffer (20 mM HEPES, pH 7.9; 1.5 mM MgCl2; 20 mM KCl; 0.2 mM EDTA; 25% glycerol (v/v); 0.2 mM PMSF; 0.5 mM DTT) was added to the orange-caped tubes (50 ml) and the pellet was completely resuspended. 1/2 PNV of high salt buffer (20 mM HEPES, pH 7.9; 0.5 mM MgCl2; 1.5 M KCl; 0.2 mM EDTA; 25% glycerol (v/v); 0.2 mM PMSF; 0.5 mM DTT) was then added, and the tube was rotating for 90 minutes in the cold room. The mixture was poured into 30 ml Corning tubes and centrifuged at 10K for 30 minutes in an SS34 rotor. The supernatant, which constitutes the nuclear extract, was pipetted into dialysis tubing and dialyzed for 2 hours in 2 L of buffer (20 mM HEPES, pH 7.9; 100 mM KCl; 0.2 mM EDTA; 25% glycerol (v/v); 0.2 mM PMSF; 0.5 mM DTT). The buffer was changed and dialysis was continued for another 2 hours. The nuclear extract was pipetted into 30 ml Corning tubes, centrifuged at 10K for 20 minutes in a SS34 rotor. The supernatant was removed and aliquoted into 1 ml aliquots. The tubes were snap frozen in liquid nitrogen, and then stored at −80°.

Spliceosome complexes were formed on the two RNA affinity substrates by combining the following ingredients: 192 μl RTP; 96 μl ATP (12.5 mM); 96 μl MgCl (80 mM); 96 μl Creatine phosphate 0.5M; 720 μl SDB; 480 μl H2O; 720 μl nuclear extract (Total 2400 μl).

These ingredients were mixed in a 50 ml orange-cap tube and incubated at 30° C. for about 40 minutes to obtain all spliceosome-associated proteins from spliceosomal complexes E, A, B, C, and spliced mRNA complexes. The reaction was then loaded onto a Sephacryl S-500 gel filtration column (50×1.5 cm) (8 μl of the reaction was kept for total RNA checking). Gel filtration was run as described herein, by collecting 1.0 ml fractions. Fractions from No. 25 to No. 80 were counted and a profile was drawn based on cpm. All peaks corresponding to a complex were pooled.

Amylose resin (50% v/v) (available from NEB, #300-21s) slurry was added at 30-60 μl/ml fractions (the actual bead volume was around 15 μl-30 μl/ml fractions) and the mixtures were rotated at 4° C. for 4 hours. The amylose resin was prewashed with 1×PBS, 10 volume×3, and resuspended in 1:1 volume of 1×PBS. After incubation with the elutions, the amylose resin was washed 3 to 5 times with 1×FSP (10 ml). 10×FSP consists of 20 mM HEPES pH 7.9; 60 mM NaCl; 0.5 mM EDTA; 0.1% Triton; 0.01% NaN3. The resin mixture was then transferred to a 1.5 ml tube and the extra liquid was discarded.

300 μl 1×maltose elution buffer was added and the mixture was rotated at 4° C. for 30-60 minutes. 10×Maltose Elution Buffer consists of 20 mM HEPES pH 7.9; 60 mM NaCl; 10 mM beta-mercaptoethanol; 12 mM Maltose (1 mM PMSF and 0.1 U/μl RNasin are optional). The mixture was briefly centrifuged and the supernatant constituted the first elution. The above steps were repeated to get the second elution. 75% of the complexes were eluted in the first elution and 15% of the complexes in the second elution.

The protein were precipitated as follows for protein identification. 30 μl of 20%SDS, 3 μl 2M DTT, 3 μl of glycogen were added to 300 μl of elution and the solution was heated in a 70° C. water bath for 5 minutes. The solution was then mixed with 1.2 ml of acetone and spinned at room temperature for 20 minutes.

The precipitated proteins were separated by 10% SDS-PAGE and letting the dye run 2.5 cm into the gel. The gel was stained with Commassie blue.

Gel slices with protein spots were isolated and subjected to digestion with Trypsin. The tryptic peptides were detected, isolated, and fragmented in a completely automated fashion on an LCQ-DECA ion trap mass spectrometer (Thermo Finnigan, San Jose, Calif.). All MS/MS spectra were searched against the National Cancer Institute (NCI) database.

The proteins identified in the spliceosomes are set forth in Tables 1, listing all spliceosome associated proteins (SAPs) that were known, and Table 2, listing putative novel SAPs. The tables are attached at the end of the application. These proteins were identified both in the spliceosomes formed on pAdL and Ftz pre-mRNA. Two of the novel SAPs has recently been shown to cause Retinitis Pigmentosa (Mol. Cell 8:375-381, 2001 and Human Mol. Genetics (2002) 11:87). The discovery of this protein as well as the other not previously known SAPs leads to the preparation of diagnostics and therapeutics.

### EQUIVALENTS

Those skilled in the art will recognize, or be able to ascertain using no more than routine experimentation, many equivalents of the specific embodiments of the invention described herein. Such equivalents are intended to be encompassed by the following claims.

TABLE 1

| | | | MS/MS | | | | | MS/MS | | | | | |
|---|---|---|---|---|---|---|---|---|---|---|---|---|---|
| | | | from AdML | | | | | from Ftz | | | | | |
| | | | s'some | | | | | S′some | | | | | |
| | Known SAPs | | | Total | Unique | Filtered | Filtered | | Total | Unique | Filtered | Filtered | NC/annotation/Gen Bank |
| Reference with link | Protein Name | Mass | Avg × Corr | peptides | sequences | peptides | unique | Avg × Corr | peptides | sequences | peptides | unique | Accession/Other Names |
| Sm core proteins | | | | | | | | | | | | | |
| SW:SMD1_HUMAN | Sm D1 | 13282 | 4.081 | 114 | 9 | 83 | 4 | 3.977 | 6 | 3 | 8 | 3 | P13641 |
| SW:SMD2_HUMAN | Sm D2 | 13527 | 3.897 | 50 | 14 | 26 | 5 | 4.012 | 15 | 8 | 10 | 5 | P43330 |
| SW:SMD3_HUMAN | Sm D3 | 13916 | 3.859 | 19 | 7 | 10 | 2 | 4.169 | 11 | 5 | 4 | 2 | P43331 |
| SW:RUXG_HUMAN | Sm G | 8496 | 2.865 | 7 | 2 | 7 | 2 | 3.171 | 1 | 1 | 1 | 1 | Q15357 |
| SW:RSMB_HUMAN | Sm B/B′ | 24610 | 3.222 | 16 | 9 | 4 | 3 | 3.288 | 8 | 5 | 4 | 2 | P14678 |
| SW:RUXE_HUMAN | Sm E | 10804 | 3.204 | 5 | 4 | 4 | 3 | 2.578 | 4 | 3 | 3 | 2 | P08578 |
| SW:RUXF_HUMAN | Sm F | 9725 | 2.799 | 25 | 13 | 3 | 2 | 2.731 | 16 | 6 | 4 | 3 | Q15356 |
| U6 snRNA-associated | | | | | | | | | | | | | |
| sm-like proteins | | | | | | | | | | | | | |
| SWN:LSM4_HUMAN | Ism4 | 15350 | 2.677 | 11 | 3 | 7 | 2 | 2.759 | 7 | 2 | 4 | 2 | Q9y4z0 |
| SWN:LSM2_HUMAN | Ism2 | 10835 | 4.074 | 9 | 6 | 4 | 2 | 3.435 | 3 | 2 | 3 | 2 | Q9y333 |
| SWN:LSM7_HUMAN | Ism7 | 11602 | 2.498 | 3 | 2 | 3 | 2 | *** | | | | | Q9uk45 |
| SWN:LSM6_HUMAN | Ism6 | 9128 | 2.989 | 4 | 4 | 1 | 1 | 2.338 | 2 | 1 | 2 | 1 | Q9y4y8 |
| SWN:LSM3_HUMAN | Ism3 | 11714 | 2.24 | 10 | 6 | 1 | 1 | 2.457 | 6 | 2 | 3 | 1 | Q9y4z1 |
| SWN:LSM5_HUMAN | Ism5 | 9806 | *** | | | | | *** | | | | | Q9Y4Y9 |
| SWN:LSM8_HUMAN | Ism8 | 10271 | *** | | | | | *** | | | | | O95777 |
| U1 snRNP | | | | | | | | | | | | | |
| specific proteins | | | | | | | | | | | | | |
| SW:RU17_HUMAN | U1 70k | 70081 | 3.291 | 14 | 6 | 6 | 2 | 3.246 | 3 | 3 | 1 | 1 | P08621 |
| SW:RU1C_HUMAN | U1 C | 17394 | 3.771 | 2 | 1 | 2 | 1 | 3.853 | 2 | 2 | 1 | 1 | P09234 |
| SW:RU1A_HUMAN | U1 A | 31280 | 2.208 | 7 | 5 | 1 | 1 | 2.775 | 7 | 5 | 2 | 2 | P09012 |
| U2 snRNP | | | | | | | | | | | | | |
| specific proteins | | | | | | | | | | | | | |
| GP:AF054284_1 | SAP155 | 145815 | 3.475 | 124 | 43 | 64 | 23 | 2.743 | 52 | 26 | 29 | 15 | |
| SW:S145_HUMAN | SAP145 | 97657 | 3.228 | 67 | 33 | 30 | 12 | 3.084 | 33 | 20 | 15 | 8 | Q13435 |
| SW:S114_HUMAN | SAP 114 | 88886 | 3.549 | 57 | 30 | 29 | 14 | 3.042 | 37 | 22 | 19 | 11 | Q15459 |
| GP:AJ001443_1 | SAP 130 | 135592 | 3.471 | 16 | 10 | 9 | 5 | 3.096 | 9 | 6 | 7 | 5 | |
| PIR2:A55749 | SAP61 | 58849 | 3.308 | 33 | 21 | 20 | 11 | 2.921 | 15 | 7 | 7 | 3 | |
| SW:SP62_HUMAN | SAP 62 | 49196 | 2.992 | 12 | 7 | 8 | 4 | 2.24 | 7 | 6 | 1 | 1 | Q15428 |
| SW:SP49_HUMAN | SAP49 | 44386 | 3.543 | 7 | 4 | 4 | 2 | 4.207 | 5 | 5 | 2 | 2 | Q15427 |
| SW:RU2A_HUMAN | U2 A′ | 28444 | 3.25 | 39 | 19 | 19 | 9 | 2.863 | 18 | 11 | 12 | 8 | P09661 |
| SW:RU2B_HUMAN | U2 B″ | 25486 | 4.024 | 11 | 6 | 6 | 1 | 5.28 | 6 | 3 | 1 | 1 | P08579 |
| AAK94041 | p14 | 14584 | *** | | | | | *** | | | | | |
| U5 snRNP | | | | | | | | | | | | | |
| specific proteins | | | | | | | | | | | | | |
| SWN:U520_HUMAN | U5-200 | 194479 | 3.395 | 150 | 62 | 101 | 36 | 2.929 | 154 | 56 | 96 | 32 | lost link, DEIH-box |
| GP:AB007510_1 | U5-220 | 273785 | 3.037 | 111 | 42 | 59 | 25 | 2.778 | 143 | 53 | 77 | 29 | lost link |
| GPN:BC002360_1 | U5-116 | 109478 | 3.308 | 70 | 38 | 47 | 22 | 2.812 | 68 | 34 | 45 | 16 | EF-2-like |
| GPN:BC001666_1 | U5-102 | 106925 | 3.046 | 6 | 5 | 4 | 4 | 2.744 | 9 | 6 | 3 | 3 | hPrp6, hPrp1, SPF107 |
| GPN:BC002366_1 | U5-100 | 95583 | 2.783 | 27 | 21 | 17 | 12 | 2.474 | 22 | 14 | 13 | 10 | DEAD-box |
| GP:AF090988_1 | U5-40 | 39299 | 3.865 | 4 | 3 | 3 | 2 | 3.169 | 6 | 3 | 5 | 3 | |
| SW:DIM1_HUMAN | U5-15 | 16786 | 3.649 | 3 | 3 | 1 | 1 | 2.247 | 1 | 1 | 1 | 1 | O14834, dim1 homolog |
| U4/U6.U5 snRNP | | | | | | | | | | | | | |
| specific proteins | | | | | | | | | | | | | |
| PIR2:T50839 | U4/U6-90k | 77529 | 3.335 | 15 | 13 | 9 | 8 | 2.558 | 11 | 8 | 6 | 5 | hPrp3 |
| GPN:BC007424_1 | U4/U6-60k | 58321 | 3.037 | 10 | 8 | 6 | 5 | 2.852 | 10 | 7 | 6 | 4 | hPrp4 |
| GP:AF083385_1 | SPF30 | 26711 | 3.46 | 13 | 7 | 7 | 3 | 2.836 | 5 | 3 | 4 | 2 | |
| PIR2:T000034 | Tri-snRNP 110k | 90255 | 3.147 | 22 | 16 | 11 | 11 | 2.918 | 19 | 15 | 11 | 9 | SPF90/SART1/hSnu66 |
| GP:AF353989_1 | Tri-snRNP 65k | 65145 | 2.46 | 2 | 2 | 1 | 1 | *** | | | | | hSad1p |
| SW:NHPX_HUMAN | Tri- | 14174 | 2.926 | 7 | 4 | 5 | 3 | 2.861 | 2 | 2 | 1 | 1 | P55769, AF155235, |
| | snRNP15 5kD | | | | | | | | | | | | hSnu13p |
| PIR1:S64705 | CyP-60 | 58823 | 3.089 | 12 | 9 | 11 | 8 | 3.194 | 1 | 1 | 1 | 1 | cyclophilin-like protein CyP- |
| | | | | | | | | | | | | | 60 |
| GPN:AF271652_1 | PPIL3b | 18155 | 3.162 | 9 | 6 | 5 | 4 | 3.185 | 5 | 5 | 2 | 2 | cyclophilin-like protein |
| | | | | | | | | | | | | | PPIL3b (PPIL3) |
| Step II proteins | | | | | | | | | | | | | |
| SW:PR16_HUMAN | hPrp16 | 140473 | 3.054 | 7 | 6 | 6 | 5 | 2.569 | 5 | 3 | 3 | 2 | Q92620, DEAH-box |
| GP:AF038392_1 | hPrp17 | 65521 | 3.2 | 10 | 6 | 6 | 4 | 2.585 | 3 | 2 | 1 | 1 | |
| GPN:BC010634_1 | hSlu7 | 68343 | 3.448 | 6 | 6 | 2 | 2 | 2.636 | 4 | 4 | 2 | 2 | |
| GPN:BC000794_1 | hPrp18 | 39860 | 4.918 | 1 | 1 | 1 | 1 | *** | | | | | |
| SW:DDX8_HUMAN | hPrp22 | 139315 | 3.364 | 12 | 9 | 10 | 7 | 2.71 | 15 | 13 | 9 | 8 | Q14562, HRH1, DEAH- |
| | | | | | | | | | | | | | box |
| mRNP proteins | | | | | | | | | | | | | |
| SWN:RBM8_HUMAN | Y14 | 19889 | 2.842 | 7 | 5 | 5 | 3 | 2.461 | 5 | 4 | 3 | 2 | Q9y5s9, RBM8 |
| AF047002 | Aly | 28861 | 3.32 | 8 | 6 | 2 | 1 | 2.841 | 9 | 6 | 3 | 2 | NM_005782, BEF |
| SW:MGN_HUMAN | magoh | 17164 | 2.934 | 7 | 5 | 3 | 2 | 2.713 | 7 | 4 | 5 | 3 | P50606 |
| PIR2:JC4525 | RNPS1 | 34208 | 4.132 | 7 | 5 | 3 | 2 | 3.191 | 4 | 3 | 3 | 2 | RHA-binding protein E5 1 |
| SR proteins | | | | | | | | | | | | | |
| SW:SFR1_HUMAN | SF2/ASF | 27613 | 2.828 | 27 | 14 | 19 | 10 | 2.852 | 42 | 12 | 36 | 9 | Q07955 |
| SW:SFR3_HUMAN | SRp20 | 19330 | 2.762 | 16 | 4 | 11 | 3 | 2.764 | 11 | 4 | 8 | 3 | P23152 |
| SW:SFR7_HUMAN | 9G8 | 27367 | 2.529 | 11 | 4 | 7 | 3 | 2.416 | 6 | 3 | 4 | 3 | Q16629 |
| SW:SFR6_HUMAN | SRp55 | 39568 | 3.181 | 6 | 4 | 4 | 3 | 3.027 | 8 | 4 | 6 | 3 | Q13247 |
| SW:SFR4_HUMAN | SRp75 | 56792 | 2.381 | 5 | 4 | 3 | 2 | 2.537 | 6 | 3 | 2 | 2 | Q08170 |
| SW:SFR5_HUMAN | SRp40 | 31264 | 3.327 | 2 | 2 | 2 | 2 | 3.018 | 4 | 1 | 4 | 1 | Q13243 |
| SW:SFR9_HUMAN | SRp30c | 25542 | 2.759 | 5 | 3 | 2 | 2 | 2.414 | 10 | 6 | 5 | 4 | Q13242 |
| SW:SFR2_HUMAN | SC35 | 25575 | 2.974 | 2 | 2 | 1 | 1 | *** | | | | | Q01130 |
| GP:AF048977_1 | SRm160 | 93519 | 3.559 | 5 | 4 | 2 | 2 | 3.578 | 7 | 5 | 5 | 3 | |
| SW:SFRB_HUMAN | p54 | 53542 | 3.633 | 9 | 5 | 5 | 3 | 2.276 | 2 | 2 | 1 | 1 | Q05519, SFRS11 |
| SW:SPR8_HUMAN | SWAP | 104821 | *** | | | | | 2.282 | 4 | 4 | 1 | 1 | Q12872, SFRS8 |
| Other dead box/ | | | | | | | | | | | | | |
| helicase proteins | | | | | | | | | | | | | |
| SW:HE47_HUMAN | UAP56 | 48991 | 3.131 | 7 | 7 | 3 | 3 | 2.54 | 7 | 5 | 5 | 3 | hBAT1, p47, HE47, Q13838 |
| SW:DD15_HUMAN | hPrp43 | 92829 | 3.237 | 14 | 11 | 12 | 9 | 2.778 | 11 | 10 | 5 | 4 | O43143 |
| SW:DD17_HUMAN | | 72371 | 2.894 | 13 | 10 | 11 | 8 | 2.554 | 20 | 11 | 14 | 6 | Q92841, p72 |
| SW:DDX3_HUMAN | | 73243 | 3.238 | 6 | 5 | 6 | 5 | 2.54 | 3 | 3 | 2 | 2 | O00571, nlp2 |
| SW:DDX5_HUMAN | | 69148 | 2.643 | 10 | 7 | 6 | 6 | 2.515 | 9 | 6 | 5 | 3 | P17844, p68, mann |
| SW:DD16_HUMAN | hPrp2 | 119172 | 2.64 | 10 | 7 | 6 | 5 | 2.613 | 9 | 6 | 5 | 3 | O60231 |
| SW:DDX9_HUMAN | | 140877 | 3.063 | 7 | 6 | 2 | 1 | 2.75 | 9 | 7 | 6 | 5 | Q08211, ndh ii |
| GP:AF106680_1 | hPrp5 | 117266 | 2.539 | 2 | 2 | 1 | 1 | 3.455 | 1 | 1 | 1 | 1 | RNA helicase |
| Other SAPs | | | | | | | | | | | | | |
| SW:CB80_HUMAN | CBP 80 | 91839 | 2.846 | 28 | 16 | 20 | 12 | 2.672 | 38 | 18 | 20 | 10 | Q09161 |
| SW:CB20_HUMAN | CBP 20 | 18001 | 3.281 | 8 | 4 | 2 | 1 | 2.482 | 8 | 8 | 4 | 3 | P52298 |
| SW:U2AF_HUMAN | U2 AF65 | 53501 | 3.641 | 15 | 8 | 11 | 5 | 3.758 | 6 | 5 | 1 | 1 | P26368 |
| SW:U2AG_HUMAN | U2 AF35 | 27872 | 2.77 | 3 | 3 | 2 | 2 | 3.942 | 2 | 2 | 2 | 2 | QD1081 |
| GPN:AY029347_1 | hPrp4/kinase | 116973 | 3.838 | 6 | 3 | 5 | 2 | 2.967 | 3 | 3 | 2 | 2 | serine/threonine-protein |
| | | | | | | | | | | | | | kinase (PRP4) |
| GPN:BC008719_1 | hPrp19 | 55181 | 3.165 | 43 | 17 | 19 | 9 | 2.931 | 20 | 10 | 12 | 7 | nuclear matrix protein |
| | | | | | | | | | | | | | NMP200,WD repeat |
| GP:AL050369_1 | hPrp31 | 55424 | 3.049 | 8 | 6 | 4 | 3 | 3.207 | 4 | 4 | 3 | 3 | |
| GP:AF049523_1 | hFBP 11 | | 2.917 | 8 | 4 | 7 | 3 | 2.864 | 11 | 5 | 6 | 3 | huntingtin-interacting |
| | | | | | | | | | | | | | protein HYPA/FBP11 |
| GP:AP255443_1 | hCRN | 99201 | 3.032 | 25 | 16 | 16 | 11 | 3.318 | 8 | 6 | 4 | 4 | CGI-201, TPR-repeat |
| Y08765/Y08766 | SFI/mBBP | 68632 | *** | | | | | *** | | | | | seveal isoforms |
| SAPs with | | | | | | | | | | | | | |
| other function | | | | | | | | | | | | | |
| PIR2:T08599 | CA150 | 123960 | 2.925 | 56 | 28 | 33 | 15 | 2.684 | 32 | 23 | 21 | 13 | |
| SW:SKIP_HUMAN | skip | 61494 | 3.507 | 22 | 18 | 8 | 7 | 2.896 | 18 | 9 | 12 | 5 | snw1, nuclear receptor |
| | | | | | | | | | | | | | coactivator ncoa-62 |
| GPN:BC007871_1 | SPF45 | 44962 | 2 | 2 | 1 | 1 | | 3.118 | 1 | 1 | 1 | 1 | G-patch, RRM, DNA dmage repair |
| GP:AF083383_1 | SPF38 | 34290 | 2.876 | 4 | 3 | 2 | 2 | 3.823 | 5 | 4 | 4 | 3 | WD |
| Proteins enriched | | | | | | | | | | | | | |
| in H complex | | | | | | | | | | | | | |
| SW:PTB_HUMAN | hnRNP I/PTB | 57221 | 4.13 | 25 | 18 | 11 | 6 | 2.206 | 3 | 3 | 1 | 1 | P26599 |
| SW:ROA1_HUMAN | hnRNP A1 | 36715 | 2.48 | 15 | 11 | 8 | 6 | 3.354 | 20 | 13 | 8 | 5 | P09651 |
| SW:ROA2_HUMAN | hnRNP A2/B1 | 37430 | 3.515 | 3 | 3 | 2 | 2 | 2.653 | 14 | 8 | 6 | 4 | P22626 |
| SWN:ROR_HUMAN | hnRNP R | 70943 | 3.074 | 10 | 9 | 7 | 7 | 3.006 | 15 | 9 | 11 | 6 | O43390 |
| SW:ROC_HUMAN | hnRNP C1/C2 | 33299 | 2.828 | 7 | 6 | 6 | 6 | 2.791 | 34 | 10 | 27 | 10 | P07910 |
| SW:ROK_HUMAN | hnRNP K | 50976 | 3.482 | 5 | 5 | 5 | 5 | 3.356 | 16 | 9 | 9 | 6 | Q07244 |
| SW:ROA0_HUMAN | hnRNP A0 | 30841 | 4.284 | 7 | 3 | 4 | 2 | 4.373 | 3 | 2 | 3 | 2 | Q13151 |
| SW:ROH1_HUMAN | hnRNP H | 49229 | 3.47 | 6 | 5 | 4 | 3 | *** | | | | | P31943 |
| SW:ROL_HUMAN | hnRNP L | 60187 | 2.884 | 6 | 6 | 3 | 3 | 2.989 | 55 | 21 | 37 | 14 | P14866 |
| SWN:ROD_HUMAN | hnRNP D0 | 38434 | 2.879 | 4 | 3 | 3 | 2 | 3.365 | 8 | 5 | 4 | 1 | Q14103 |
| SW:ROM_HUMAN | hnRNP M | 77489 | 2.815 | 5 | 5 | 3 | 3 | *** | | | | | P52272 |
| GPN:BC001616_1 | hnRNP A/B | 30588 | 3.667 | 2 | 1 | 2 | 1 | 3.06 | 1 | 1 | 1 | 1 | |
| SW:ROA3_HUMAN | hnRNP A3 | 39686 | 3.655 | 6 | 5 | 2 | 2 | 2.823 | 7 | 6 | 3 | 2 | P51991 |
| SW:ROF_HUMAN | hnRNP F | 45672 | 3.429 | 2 | 2 | 2 | 2 | *** | | | | | P52597 |
| SW:ROG_HUMAN | hnRNP G | 42404 | *** | | | | | 3.036 | 10 | 4 | 7 | 3 | P38159 |
| PIR2:B54857 | NF-AT 90k | 73339 | 3.117 | 7 | 4 | 7 | 4 | 2.919 | 9 | 8 | 7 | 6 | |
| PIR2:A54857 | NF-AT 45k | 44697 | 3.436 | 7 | 6 | 4 | 4 | 3.197 | 8 | 7 | 6 | 5 | |
| GP:AF037448_1 | Gry-rbp | 69633 | 3.882 | 3 | 3 | 1 | 1 | 3.149 | 9 | 5 | 6 | 4 | RRM RNA binding protein |
| GPN:BC008875_1 | PUF60 | 50171 | 3.316 | 57 | 29 | 24 | 13 | 3.217 | 29 | 18 | 5 | 4 | siah binding protein 1, FBP |
| | | | | | | | | | | | | | interacting repressor, PTB |

TABLE 2

| | | | MS/MS | | | | | MS/MS | | | | |
|---|---|---|---|---|---|---|---|---|---|---|---|---|
| | Putative Novel SAPS | | AdML | | | | | from Ftz | | | | |
| | NCI annotation | | s'some | Total | Unique | Filtered | Filtered | S'some | Total | Unique | Filtered | Filtered |
| Reference with link | Potientially new SAPs | Mass | Avg × Corr | peptides | sequences | peptides | unique | Avg × Corr | peptides | sequences | peptides | unique |
| GP:AF356524_1 | nuclear receptor transcription cofactor (SHARP) | 402248 | 3.39 | 32 | 25 | 16 | 13 | 2.829 | 23 | 18 | 10 | 8 |
| GPN:BC007208 1 | HCNP,XPA-binding protein 2 | 100010 | 3.241 | 27 | 18 | 17 | 12 | 2.779 | 9 | 8 | 6 | 5 |
| GP:AC004858_3 | U1 small nbonucleoprotein 1SNRP homolog, | 94122 | 3.331 | 50 | 26 | 33 | 15 | 3.454 | 24 | 15 | 13 | 8 |
| | Pole(A) binding protein | | | | | | | | | | | |
| PIR2:155595 | splicing factor | 58657 | 3.346 | 32 | 14 | 18 | 7 | 2.9 | 10 | 5 | 4 | 2 |
| GP:AB034205_1 | cisplatin resistance-associated overexpressed | 51466 | 3.45 | 14 | 9 | 10 | 6 | 3.149 | 8 | 7 | 7 | 6 |
| | protein | | | | | | | | | | | |
| SW:BUB3_HUMAN | mitotic checkpoint protein bub3 | 37155 | 2.982 | 6 | 5 | 6 | 5 | 2.956 | 7 | 4 | 5 | 3 |
| SWN:ELV1_HUMAN | elav-like protein 1 (hu-antigen r) (hur) | 36062 | 2.787 | 2 | 2 | 1 | 1 | 2.93 | 10 | 6 | 5 | 3 |
| GPN:BC001621 1 | Npw38-binding protein NpwBP | 69998 | 2.64 | 11 | 9 | 7 | 5 | 2.332 | 5 | 4 | 2 | 1 |
| SW:RED_HUMAN | red protein (rer protein) (ik factor) (cytokine ik) | 65630 | 2.781 | 9 | 7 | 5 | 4 | 2.517 | 4 | 4 | 2 | 2 |
| SW:Z207_HUMAN | O43670, zinc finger protein 207 | 50751 | 3.199 | 10 | 6 | 2 | 2 | 2.64 | 6 | 5 | 1 | 1 |
| SW:YB1_HUMAN | y box binding protein-1 (yb-1) (ccaat-binding | 35924 | 4.636 | 10 | 9 | 1 | 1 | 4.316 | 6 | 5 | 2 | 1 |
| | transcription factor i subunit a) (cbf-a) enhancer | | | | | | | | | | | |
| | factor | | | | | | | | | | | |
| GPN:BC0003376_1 | ELAV (embryonic lethal, abnormal vision, | 36092 | 3.878 | 2 | 1 | 1 | 1 | 3.862 | 5 | 1 | 3 | 1 |
| | Drosophila)-like 1 (Hu antigen R) | | | | | | | | | | | |
| PIR2:A53545 | nuclear matrix protein p84 | 75627 | 3.557 | 13 | 10 | 6 | 5 | 2.99 | 29 | 13 | 15 | 7 |
| GP:AF155096 1 | NY-REN-6 antigen, partial cds | | 2.973 | 17 | 6 | 4 | 2 | 2.82 | 11 | 4 | 6 | 3 |
| SW:IF4N HUMAN | eukaryotic initiaion factor 4a-like nuk-34 | 46833 | 3.147 | 14 | 12 | 9 | 9 | 2.383 | 11 | 8 | 6 | 5 |
| SWN:CRK7_HUMAN | cell division cycle 2-related protein kinase 7 | 164155 | 3.146 | 11 | 8 | 6 | 3 | 3.07 | 7 | 6 | 5 | 4 |
| | (cdc2-related protein kinase 7) (crkrs) | | | | | | | | | | | |
| GPN:BC001403 1 | pre-mRNA cleavage factor Im (25kD) | 26227 | 3.246 | 11 | 7 | 7 | 5 | 3.511 | 6 | 5 | 4 | 3 |
| GP:AF044333 1 | pleiotropic regulator 1 (PLRG1) | 57194 | 3.768 | 13 | 10 | 7 | 6 | 3.656 | 4 | 4 | 3 | 3 |
| SW:G10_HUMAN | P41223, human homolog of xenopus maternal | 16844 | 2.277 | 2 | 2 | 1 | 1 | 2.431 | 4 | 2 | 3 | 1 |
| | g10 protein (edg-2) | | | | | | | | | | | |
| GP:AJ271745_1 | double-stranded RNA binding nuclear protein | 76033 | 2.804 | 2 | 1 | 1 | 1 | 2.41 | 5 | 3 | 3 | 1 |
| | DBRP76 (ILF3 gene) | | | | | | | | | | | |
| GP:AJ276706_1 | partial mRNA for WTAP (wilm's tumor | | 3.572 | 2 | 1 | 1 | 1 | 3.715 | 2 | 2 | 2 | 2 |
| | associating protein) (hFL2D) | | | | | | | | | | | |
| GP:AJ279080_1 | putative transcription factor (ORF1), ORF1 S1 | 104804 | 3.544 | 3 | 3 | 3 | 3 | 3.033 | 3 | 3 | 2 | 2 |
| | RNA binding protein | | | | | | | | | | | |
| SW:GCFC_HUMAN | gc-rich sequence dna-binding factor homolog, | 29010 | 3.167 | 3 | 3 | 3 | 3 | 2.655 | 2 | 1 | 2 | 1 |
| | Q9y5b6 | | | | | | | | | | | |
| GP:U70667_1 | Fas-ligand associated factor 1(FLAF1), | | 2.51 | 10 | 6 | 3 | 1 | 2.616 | 5 | 4 | 2 | 2 |
| | WWP/WW motif | | | | | | | | | | | |
| GNP:BC005152_1 | similar to mouse GIt3 or D. malanogaster | 22774 | 3.154 | 1 | 1 | 1 | 1 | 2.606 | 2 | 2 | 2 | 2 |
| | transcription factor IIB | | | | | | | | | | | |
| SWN:RB56_HUMAN | Q92804, tata-binding protein associated factor | 61830 | 2.726 | 3 | 2 | 3 | 2 | 2.011 | 3 | 2 | 2 | 1 |
| | 2n (ma-binding protein 56) (tafii68) | | | | | | | | | | | |
| SWN:CYCK_HUMAN | O75909, cyclin k | 41293 | 3.653 | 2 | 1 | 1 | 1 | 4.355 | 2 | 2 | 1 | 1 |
| GPN:BC003015_1 | Similar to expressed sequence 2 embryonic | | 3.363 | 3 | 3 | 3 | 3 | 4.199 | 1 | 1 | 1 | 1 |
| | lethal | | | | | | | | | | | |
| GP:AB016088 1 | RNA binding protein, partial cds | | 3.764 | 10 | 5 | 5 | 2 | 4.054 | 3 | 3 | 1 | 1 |
| SW:SP18_HUMAN | O00422, sin3 associated polypeptide p18 | 17561 | 3.414 | 2 | 2 | 1 | 1 | 3.435 | 5 | 5 | 1 | 1 |
| GPN:BC002548 1 | Simular to Moloney leukemia virus 10 | 113671 | 3.215 | 4 | 4 | 2 | 2 | 3.434 | 2 | 2 | 1 | 1 |
| PIR2:I38191 | nucleic acid binding protein (fragment) | | 4.65 | 1 | 1 | 1 | 1 | 3.364 | 2 | 2 | 1 | 1 |
| SW:CIRP_HUMAN | cold-inducible ma-binding protein (glycine-rich | 18648 | 3.037 | 5 | 3 | 4 | 2 | 3.349 | 2 | 2 | 1 | 1 |
| | ma-binding protein crp) | | | | | | | | | | | |
| GP:AF112222 1 | nuclear protein SDK3 | 81584 | 3.026 | 4 | 3 | 1 | 1 | 3.311 | 5 | 3 | 1 | 1 |
| GP:L76159_1 | Facioscapulohumeral muscular dystrophy | 29172 | 3.363 | 2 | 1 | 2 | 1 | 3.079 | 1 | 1 | 1 | 1 |
| | region gene-1 | | | | | | | | | | | |
| SW:GR78_HUMAN | 78 kda glucose-regulated protein precursor (grp | 72116 | 4.29 | 3 | 2 | 1 | 1 | 2.955 | 1 | 1 | 1 | 1 |
| | 78) (immunogobulin heavy chain binding | | | | | | | | | | | |
| | protein) (bip) | | | | | | | | | | | |
| GPN:BC000495_1 | CD2 antigen (cytoplasmic tail)-binding protein 2 | 37646 | 3.374 | 4 | 3 | 2 | 2 | 2.887 | 4 | 4 | 1 | 1 |
| SW:MFA1_HUMAN | P55081, microfibrillar-associated protein 1 | 51855 | 2.687 | 6 | 5 | 3 | 3 | 2.777 | 3 | 3 | 1 | 1 |
| GP:AF015044_1 | EH-binding protein, binds EH domains of eps 15 | | 3.132 | 6 | 6 | 3 | 3 | 2.771 | 1 | 1 | 1 | 1 |
| GPN:BCO10381 1 | nuclear matriz protein p84 | 75666 | 2.555 | 5 | 5 | 3 | 3 | 2.768 | 6 | 4 | 1 | 1 |
| GP:AF361746_1 | endothelial cell-selective adhesein molecule | 41208 | 3.24 | 3 | 1 | 3 | 1 | 2.555 | 1 | 1 | 1 | 1 |
| | (ESAM), immunoglobulin superfamily; contains | | | | | | | | | | | |
| | V and C2 domains | | | | | | | | | | | |
| GP:AJ271741_1 | partial ILF3 gene for interleukin enhancer | 25972 | 2.303 | 3 | 2 | 2 | 1 | 2.379 | 4 | 3 | 1 | 1 |
| | binding factor 3 | | | | | | | | | | | |
| SW:EF11_HUMAN | elongation factor 1-alpha 1 (ef-1-alpha-1) | 50141 | 2.852 | 6 | 5 | 5 | 4 | 2.377 | 1 | 1 | 1 | 1 |
| | (elongation factor tu) (ef-tu) | | | | | | | | | | | |
| SW:DBPA_HUMAN | dna-bincsng protein a (cold shock domain | 40060 | 3.194 | 10 | 8 | 5 | 3 | 2.374 | 2 | 2 | 1 | 1 |
| | protein a) (single-strand dna binding protein nf- | | | | | | | | | | | |
| | gmb) | | | | | | | | | | | |
| | Hypothetical pew SAPs | | | | | | | | | | | |
| PIR2:T00365 ?? | hypothetical protein KIAA0670 (fragment) | | 3.207 | 77 | 37 | 44 | 18 | 3.001 | 72 | 33 | 45 | 17 |
| PIR2:T02345 | hypothetical protein KIAA0324 (fragment), | | 3.069 | 58 | 30 | 31 | 17 | 2.885 | 37 | 25 | 20 | 13 |
| | SRm300, matrx2 related | | | | | | | | | | | |
| SW:Y017 HUMAN | hypothetical protein kiaa0017 | 44606 | 3.668 | 39 | 20 | 24 | 9 | 3.192 | 19 | 11 | 13 | 5 |
| GP:AB007892 1 | KIAA0432 mRNA | 56171 | 3.417 | 51 | 32 | 23 | 16 | 2.962 | 27 | 21 | 15 | 12 |
| PIR2:T00333 | hypothetical protein KIAA0560 | 163986 | 3.292 | 31 | 21 | 18 | 12 | 2.643 | 30 | 19 | 17 | 12 |
| PIR2:T12455 | hypothetical protein DKFZp564H2023 1 | | 2.898 | 35 | 18 | 20 | 10 | 2.931 | 67 | 21 | 40 | 10 |
| | (fragments) | | | | | | | | | | | |
| SW:YS64_HUMAN | hypothetical protein s164 (fragment) | | 3.614 | 12 | 6 | 7 | 4 | 2.998 | 16 | 7 | 12 | 6 |
| GP:AK023659_1 | clone PLACE1009798, weakly similar to RLR1 | 73795 | 2.902 | 12 | 8 | 11 | 7 | 2.698 | 33 | 16 | 25 | 12 |
| | PROTEIN | | | | | | | | | | | |
| PIR2:T46386 | hypothetical protein DKFZp434P011 1 | | 3.071 | 15 | 11 | 11 | 8 | 2.602 | 17 | 12 | 12 | 9 |
| | (fragment) | | | | | | | | | | | |
| GPN:BC003118_1 | clone MGC-2655 IMAGE 3537243, mRNA, | 34849 | 3.259 | 5 | 3 | 4 | 2 | 2.565 | 14 | 6 | 11 | 4 |
| | complete cds | | | | | | | | | | | |
| GP:AF151059 1 | HSPC225 (FBP11 related) | 24297 | 2.938 | 14 | 6 | 8 | 5 | 2.66 | 15 | 5 | 10 | 4 |
| PIR2:139463 | gene anonymous protein - human | 78536 | 3.161 | 5 | 4 | 3 | 2 | 2.629 | 16 | 12 | 8 | 6 |
| SWN:CG80_HUMAN | hypothetical protein cgi-110 (protein hspc175 | 14585 | 3.8 | 13 | 7 | 7 | 2 | 3.082 | 5 | 4 | 4 | 3 |
| GPN:BC006397_1 | Similar to hypothetical protein FLJ12479, clone | 102135 | 3.052 | 9 | 5 | 4 | 2 | 2.882 | 7 | 6 | 4 | 3 |
| | MGC:13150 IMAGE:4298786 | | | | | | | | | | | |
| GP:AL031668_1 | Human DNA sequence from clone RP1-64K7 | 32214 | 2.707 | 2 | 2 | 1 | 1 | 2.547 | 5 | 3 | 4 | 2 |
| | on chromosome 20q11 21-11 23 Contains the | | | | | | | | | | | |
| | EIF2S2 gene for eukaryotic translation initiation | | | | | | | | | | | |
| | factor 2 subunit 2 (beta, 38kD), a putative novel | | | | | | | | | | | |
| | gene, the gene for heterogenous nuclear | | | | | | | | | | | |
| | ribonucleoprotein RALY or aut | | | | | | | | | | | |
| GP:D38552_1 | KIAA0073 gene, partial cds, The ha1539 protein | | 2.924 | 14 | 10 | 9 | 7 | 2.585 | 8 | 6 | 4 | 4 |
| | is retated to cyclophilin | | | | | | | | | | | |
| GP:AB046824_1 | Homo sapiens mRNA for KIAA1604 protein, | | 3.596 | 16 | 11 | 11 | 8 | 3.345 | 8 | 6 | 3 | 3 |
| | partial cds, Start codon is not identified | | | | | | | | | | | |
| GP:AF130096_1 | FLC0586 PRO2855 mRNA, complete cds | 34655 | 3.323 | 10 | 4 | 8 | 3 | 3.174 | 3 | 2 | 3 | 2 |
| GP:BC003048_1 | CGI-124 protein | 18237 | 3.038 | 8 | 4 | 7 | 4 | 2.716 | 4 | 3 | 3 | 2 |
| GP:AB018344_1 | Homo sapiens mRNA for KIAA0801 protein, | 117461 | 3.37 | 24 | 14 | 14 | 9 | 2.586 | 10 | 9 | 3 | 2 |
| | complete cds | | | | | | | | | | | |
| GP:AK027098_1 | Homo sapiens cDNA -FL23445 fis, clone | 23671 | 2.498 | 4 | 3 | 3 | 2 | 2.566 | 10 | 6 | 3 | 3 |
| | HS101721, unnamed protein product | | | | | | | | | | | |
| GPN:BC003402_1 | hypothetical protein FLJ10290, clone MGC:4943 | 46896 | 3.376 | 8 | 8 | 3 | 3 | 2.5 | 5 | 4 | 3 | 2 |
| | IMAGE 3449258, mRNA, complete cds | | | | | | | | | | | |
| PIR2:T12485 | hypothetical protein DKFZp564O2082.1- | 28722 | 4.171 | 4 | 4 | 1 | 1 | 3.224 | 6 | 4 | 2 | 2 |
| | human | | | | | | | | | | | |
| GPN:BC004442_1 | Similar to RIKEN cDNA 5830446M03 gene, | 32992 | 2.902 | 4 | 4 | 3 | 3 | 3 | 2 | 1 | 2 | 1 |
| | clone MGC 4036 IMAGE 2820683, mRNA, | | | | | | | | | | | |
| | complete | | | | | | | | | | | |
| PIR2:T17232 | hypothetical protein DKFZp434l116 1 - human | | 3.077 | 4 | 4 | 2 | 2 | 2.956 | 4 | 4 | 2 | 2 |
| | (fragment) | | | | | | | | | | | |
| GPN:BC006474_1 | clone IMAGE 2820942, mRNA, partial cds | | 3.342 | 2 | 2 | 2 | 2 | 2.813 | 4 | 4 | 2 | 2 |
| GPN:BC004122_1 | Similar to RIKEN cDNA 5830446M03 gene, | 10870 | 3.599 | 2 | 2 | 1 | 1 | 2.707 | 2 | 1 | 2 | 1 |
| | clone MGC:11203 IMAGE 3927759, mRNA | | | | | | | | | | | |
| SW:Y105 HUMAN | hypothetical protein kiaa0105 | 17801 | 3.049 | 3 | 3 | 3 | 3 | 2.563 | 2 | 1 | 2 | 1 |
| GPN:BC002876_1 | hypothetical protein FLJ10805 | 57544 | 3.093 | 12 | 6 | 8 | 4 | 2.561 | 3 | 2 | 2 | 1 |
| GP:AF161497_1 | Homo sapiens HSPC148 mRNA, complete cds | 26610 | 2.734 | 5 | 3 | 2 | 1 | 2.403 | 5 | 3 | 2 | 2 |
| PIR2:T12531 | hypothetical protein DKFZp4348194 1 | 96820 | 3.006 | 10 | 10 | 5 | 5 | 2.338 | 7 | 6 | 2 | 2 |
| GP:AB023146 1 | KIAA0929 protein, partial cds | | 3.205 | 22 | 16 | 15 | 11 | 2.015 | 9 | 8 | 2 | 2 |
| GPN:BC003359_1 | hypothetical protein, clone MGC 5267 | 33855 | 2.687 | 2 | 2 | 1 | 1 | 4.451 | 1 | 1 | 1 | 1 |
| | IMAGE 2900332, mRNA, complete cds | | | | | | | | | | | |
| GPN:BC000198_1 | Similar to CG11985 gene product, clone | 10135 | 3.837 | 33 | 2 | 4 | 1 | 4.05 | 2 | 1 | 1 | 1 |
| | MGC:3133 IMAGE 3392960, mRNA, complete | | | | | | | | | | | |
| | cds | | | | | | | | | | | |
| PIR:T02672 | hypothetical protein R31449_3 - human | | 3.456 | 6 | 6 | 3 | 3 | 3.604 | 4 | 4 | 1 | 1 |
| | (fragment) | | | | | | | | | | | |
| PIR2:T46935 | hypothetical protein DKFZp434D199 1 | 14263 | 3.763 | 6 | 6 | 4 | 4 | 3.106 | 1 | 1 | 1 | 1 |
| GP:AF161433 1 | HSPC315 mRNA, partial cds | | 3.043 | 3 | 2 | 2 | 1 | 2.955 | 2 | 2 | 1 | 1 |
| GP:AF132955 1 | CGI-21 protein mRNA | 37542 | 2.706 | 4 | 2 | 2 | 1 | 2.795 | 1 | 1 | 1 | 1 |
| GP:AK000741_1 | cDNA FLJ20734 fis, clone HEP08523, unnamed | 70516 | 3.219 | 3 | 3 | 1 | 1 | 2.692 | 2 | 2 | 1 | 1 |
| | protein product | | | | | | | | | | | |
| SW:Y052 HUMAN | hypothetical protein kiaa0052 (fragment) | | 2.916 | 6 | 6 | 3 | 3 | 2.611 | 3 | 3 | 1 | 1 |
| GP:AB011132 1 | KIAA0560 protein, partial cds | | 2.525 | 1 | 1 | 1 | 1 | 2.595 | 1 | 1 | 1 | 1 |
| GPN:BC004258_1 | Homo sapiens, hypothetical protein PRO1741, | 65691 | 3.813 | 3 | 2 | 2 | 2 | 2.567 | 1 | 1 | 1 | 1 |
| | clone MGC:10753 IMAGE:3347345, mRNA, | | | | | | | | | | | |
| | complete cds | | | | | | | | | | | |
| GPN:BC006350 1 | clone MGC:13125 IMAGE:4111572 | 70521 | 2.77 | 9 | 9 | 7 | 7 | 2.419 | 1 | 1 | 1 | 1 |
| GP:AL512685_1 | cDNA DKPZp547K202 (from clone | 75399 | 3.222 | 3 | 3 | 1 | 1 | 2.373 | 4 | 4 | 1 | 1 |
| | DKFZp547K202); WD-protein The frame shift | | | | | | | | | | | |
| | was determined manually | | | | | | | | | | | |
| GP:AL023804_1 | Human DNA sequence from clone RP4-633O20 | 32895 | 2.81 | 4 | 4 | 3 | 3 | 2.321 | 2 | 2 | 1 | 1 |
| | on chromosone 20q11.23-12 Contains 5′ end of | | | | | | | | | | | |
| | a gene similar to Bos taurus P14 protein (P14L), | | | | | | | | | | | |
| | ESTs. CA repeat(D20S859), STSs and GSSs, | | | | | | | | | | | |
| | complete sequence, Also similar to Drosophila | | | | | | | | | | | |
| | CG11964 protein | | | | | | | | | | | |

We claim:

1. A method for forming an isolated ribonucleoprotein complex comprising:
providing an RNA affinity substrate comprising a ribonucleoprotein assembly sequence and an affinity tag;
contacting the RNA affinity substrate with a protein mixture so as to permit the formation of a ribonucleoprotein complex on said ribonucleoprotein assembly sequence;
subjecting said ribonucleoprotein complex to chromatographic separation; and
subjecting said ribonucleoprotein complex to affinity selection, wherein the affinity tag binds to an affinity matrix;
thereby forming an isolated ribonucleoprotein complex.

2. The method of claim 1, further comprising eluting said ribonucleoprotein complex from said affinity matrix by disrupting the interaction of the affinity tag with the affinity matrix.

3. The method of claim 1, wherein said ribonucleoprotein complex is selected from the group consisting of a spliceosomal complex, an hnRNP complex, an mRNA export complex, an mRNA localization complex, an RNA editing complex, and an intron complex.

4. The method of claim 3, wherein the ribonucleoprotein complex is a spliceosomal complex selected from the group consisting of an E complex, an A complex, a B complex and a C complex.

5. The method of claim 3, the ribonucleoprotein complex is an H complex.

6. The method of claim 1, wherein the ribonucleoprotein assembly sequence is selected from the group consisting of a pre-mRNA sequence, a 5′ splice site, a 3′ splice site, and an intronless RNA.

7. The method of claim 1, wherein the affinity tag binds to an affinity matrix through the intermediate of a fusion protein comprising a polypeptide binding specifically to the affinity tag and a polypeptide binding specifically to the affinity matrix.

8. The method of claim 7, wherein the affinity tag comprises at least one MS2 or R17 coat protein recognition site and the polypeptide binding specifically to the affinity tag is an MS2 or R17 coat protein or portion thereof sufficient for binding to the MS2 or R17 coat protein recognition site.

9. The method of claim 7, wherein the polypeptide binding specifically to the affinity matrix is selected from the group consisting of a maltose binding protein; a 6× His peptide; glutathione S transferase; or portion thereof sufficient to bind specifically to an affinity matrix.

10. The method of claim 9, wherein the polypeptide binding specifically to the affinity matrix is a maltose binding protein or portion thereof sufficient to bind to amylose, the affinity matrix is an amylose matrix, and the ribonucleoprotein complex is eluted from the amylose matrix with maltose or a maltose analog.

11. The method of claim 7, comprising contacting the RNA affinity substrate with the fusion protein, such that the fusion protein binds specifically to the affinity tag prior to contacting the RNA affinity substrate with the protein mixture.

12. The method of claim 1, wherein the protein mixture is a eukaryotic cell nuclear extract or a subfraction thereof.

13. The method of claim 1, wherein the chromatographic separation is a gel filtration.

14. The method of claim 1, wherein the affinity selection is performed in a low ionic strength buffer.

15. The method of claim 14, wherein the low ionic strength buffer comprises a final salt concentration of less than about 100 mM.

16. The method of claim 1 for isolating a spliceosome comprising:
providing an RNA affinity substrate comprising a pre-mRNA sequence and an MS2 coat protein recognition site;
contacting the RNA affinity substrate with a fusion protein comprising (i) an MS2 coat protein or portion thereof sufficient to bind specifically to the MS2 coat protein recognition site and (ii) a polypeptide binding specifically to a ligand, such that the fusion protein binds to RNA affinity substrate;
contacting the RNA affinity substrate with a eukaryotic cell nuclear extract so as to permit the formation of a spliceosome mRNA complex;
subjecting the spliceosome mRNA complex to chromatographic separation; and
subjecting the spliceosome mRNA complex to affinity selection on an affinity matrix comprising the ligand,
thereby isolating a spliceosome.

17. The method of claim 16, wherein the RNA affinity substrate comprises at least two MS2 coat protein recognition sites.

18. The method of claim 16, wherein the polypeptide binding specifically to a ligand is selected from the group consisting of a maltose binding protein; a 6× His peptide; glutathione S transferase; or portion thereof sufficient to bind specifically to the ligand.

19. The method of claim 18, wherein the polypeptide binding specifically to a ligand is a maltose binding protein or portion thereof sufficient to bind to amylose; wherein the affinity selection comprises binding of the spliceosome mRNA complex on an amylose matrix and eluting the ribonucleoprotein complex from the amylose matrix with maltose or a maltose analog.

20. An isolated spliceosome preparation, isolated by the method of claim 16.

21. The isolated spliceosome preparation of claim 20, wherein more than about 10% of the pre-mRNA sequences associated with said isolated spliceosome complexes can be chased into a completely spliced mRNA in a splicing reaction.

22. The isolated spliceosome preparation of claim 20, comprising a quantitative amount of 17S U2 U2 small nuclear ribonucleoprotein (snRNP).

23. The isolated spliceosome preparation of claim 20, comprising a quantitative amount of an SP3a polypeptide.

24. The isolated spliceosome preparation of claim 20, comprising at least 90% of the proteins listed in Tables 1 and 2.

25. The isolated spliceosome preparation of claim 23, wherein said spliceosome preparation is an E complex spliceosome preparation.

26. The isolated spliceosome preparation of claim 20, wherein said spliceosome preparation is an A complex spliceosome preparation.

27. A ribonucleic acid comprising a ribonucleoprotein complex binding site and at least one phage coat protein recognition site.

28. The ribonucleic acid of claim 27, wherein the ribonucleoprotein complex binding site is a spliceosome binding site and at least one phage coat protein binding site is an MS2 or R17 coat protein recognition site.

29. The ribonucleic acid of claim 27, wherein the spliceosome binding site is an adenovirus major late pre-mRNA or a fushi tarazu pre-mRNA.

30. A nucleic acid encoding the ribonucleic acid of claim 27.

31. The nucleic acid of claim 29, operably linked to an RNA promoter capable of transcribing the nucleic acid.

32. A diagnostic assay for determining whether a subject has abnormnal ribonucleoprotein complexes, comprising:
obtaining a sample of cells from a subject;
purifying ribonucleoprotein complexes from the cells of the subject according to claim 1; and
determining the presence in the purified ribonucleoprotein complexes of one or more proteins,
wherein a difference in the amount of one or more proteins in the ribonucleoprotein complexes of the subject relative to its amount in a corresponding normal ribonucleoprotein complex indicates that the subject has abnormal ribonucleoprotein complexes.

33. The diagnostic assay of claim 33, wherein ribonucleoprotein complexes are spliceosome complexes.

34. A diagnostic assay for determining whether a subject has abnormal spliceosome complexes, comprising:
obtaining a sample of cells from a subject; and
purifying spliceosome complexes from the cells of the subject according to claim 16;
determining whether the pre-mRNA sequence was spliced during the purification,
wherein splicing of the pre-mRNA sequence indicates that the spliceosome complexes of the subject are functional, whereas the absence of splicing or the pre-mRNA indicates that the spliceosome complexes of the subject are not functional, thereby indicating that the subject has abnormal spliceosome complexes.

35. A diagnostic kit comprising at least two elements selected from the group consisting of an RNA affinity substrate; a fusion protein comprising an affinity tag binding polypeptide and a ligand binding polypeptide; a chromatographic separation reagent; and an affinity purification reagent.

36. A method for treating a subject having a disorder associated with abnormal ribonucleoprotein complexes, comprising
obtaining a sample of cells from a subject;
purifying ribonucleoprotein complexes from the cells of the subject according to claim 1;
determining the presence in the purified ribonucleoprotein complexes of one or more proteins; and
normalizing the amount of ribonucleoproteins in the subject, to thereby treat the subject having a disorder associated with abnormal ribonucleoproteins complexes.
